SEQUENCE LISTING (36 sequences)

SEQ ID NO: 1 - protein, 1807 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 2 - protein, 4 aa, Artificial Sequence
  polypeptide spacer
SGGG

SEQ ID NO: 3 - protein, 16 aa, Artificial Sequence
  polypeptide spacer
SGGGSGGGSGGGSGGG

SEQ ID NO: 4 - protein, 450 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALME

SEQ ID NO: 5 - protein, 418 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQR

SEQ ID NO: 6 - protein, 292 aa, Homo sapiens
KPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 7 - protein, 668 aa, Homo sapiens
GALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 8 - protein, 698 aa, Homo sapiens
AKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 9 - protein, 875 aa, Homo sapiens
FRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 10 - protein, 833 aa, Artificial Sequence
  synthetic construct
FRARSTSLNERPKSSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 11 - protein, 766 aa, Artificial Sequence
  synthetic construct
FRARSTSLNERPKSSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 12 - protein, 604 aa, Artificial Sequence
  synthetic construct
FRARSTSLNERPKSSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGVASFSSLYQSSCQGQLHRSVSWADSAVVMEEGSPGEVPVLVEPPGLEDVEAALGMDRRTDAYSRSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 13 - protein, 758 aa, Artificial Sequence
  synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMESGGGSGGGSGGGSGGGKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 14 - protein, 1134 aa, Artificial Sequence
  synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMESGGGSGGGSGGGSGGGGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 15 - protein, 1102 aa, Artificial Sequence
  synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRSGGGSGGGSGGGSGGGGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 16 - protein, 1309 aa, Artificial Sequence
  synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRSGGGSGGGSGGGSGGGFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 17 - protein, 1267 aa, Artificial Sequence
  synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRSGGGSGGGSGGGSGGGFRARSTSLNERPKSSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 18 - protein, 1200 aa, Artificial Sequence
  synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRSGGGSGGGSGGGSGGGFRARSTSLNERPKSSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 19 - protein, 1038 aa, Artificial Sequence
  synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRSGGGSGGGSGGGSGGGFRARSTSLNERPKSSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGVASFSSLYQSSCQGQLHRSVSWADSAVVMEEGSPGEVPVLVEPPGLEDVEAALGMDRRTDAYSRSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 20 - DNA, 2306 bp, Artificial Sequence
  synthetic construct
gctagcactagtaccatggcgaaaccgaccagcaaagatagcggcctgaaagaaaaatttaaaattctgctgggcctgggcaccccgcgcccgaacccgcgcagcgcggaaggcaaacagaccgaatttattattaccgcggaaattctgcgcgaactgagcatggaatgcggcctgaacaaccgcattcgcatgattggccagatttgcgaagtggcgaaaaccaaaaaatttgaagaacatgcggtggaagcgctgtggaaagcggtggcggatctgctgcagccggaacgcccgctggaagcgcgccatgcggtgctggcgctgctgaaagcgattgtgcagggccagggcgaacgcctgggcgtgctgcgcgcgctgttttttaaagtgattaaagattatccgagcaacgaagatctgcatgaacgcctggaagtgtttaaagcgctgaccgataacggccgccatattacctatctggaagaagaactggcggattttgtgctgcagtggatggatgtgggcctgagcagcgaatttctgctggtgctggtgaacctggtgaaatttaacagctgctatctggatgaatatattgcgcgcatggtgcagatgatttgcctgctgtgcgtgcgcaccgcgagcagcgtggatattgaagtgagcctgcaggtgctggatgcggtggtgtgctataactgcctgccggcggaaagcctgccgctgtttattgtgaccctgtgccgcaccattaacgtgaaagaactgtgcgaaccgtgctggaaactgatgcgcaacctgctgggcacccatctgggccatagcgcgatttataacatgtgccatctgatggaagatcgcgcgtatatggaagatgcgccgctgctgcgcggcgcggtgttttttgtgggcatggcgctgtggggcgcgcatcgcctgtatagcctgcgcaacagcccgaccagcgtgctgccgagcttttatcaggcgatggcgtgcccgaacgaagtggtgagctatgaaattgtgctgagcattacccgcctgattaaaaaatatcgcaaagaactgcaggtggtggcgtgggatattctgctgaacattattgaacgcctgctgcagcagctgcagaccctggatagcccggaactgcgcaccattgtgcatgatctgctgaccaccgtggaagaactgtgcgatcagaacgaatttcatggcagccaggaacgctattttgaactggtggaacgctgcgcggatcagcgcccggaaagcagcctgctgaacctgattagctatcgcgcgcagagcattcatccggcgaaagatggctggattcagaacctgcaggcgctgatggaatctggtgggggtagcggaggagggtcagggggcggcagtggaggcggaaaaccgattctgctgccgaacgaaagccagagctttgaacgcagcgtgcagctgctggatcagattccgagctatgatacccataaaattgcggtgctgtatgtgggcgaaggccagagcaacagcgaactggcgattctgagcaacgaacatggcagctatcgctataccgaatttctgaccggcctgggccgcctgattgaactgaaagattgccagccggataaagtgtatctgggcggcctggatgtgtgcggcgaagatggccagtttacctattgctggcatgatgatattatgcaggcggtgtttcatattgcgaccctgatgccgaccaaagatgtggataaacatcgctgcgataaaaaacgccatctgggcaacgattttgtgagcattgtgtataacgatagcggcgaagattttaaactgggcaccattaaaggccagtttaactttgtgcatgtgattgtgaccccgctggattatgaatgcaacctggtgagcctgcagtgccgcaaagatatggaaggcctggtggataccagcgtggcgaaaattgtgagcgatcgcaacctgccgtttgtggcgcgccagatggcgctgcatgcgaacatggcgagccaggtgcatcatagccgcagcaacccgaccgatatttatccgagcaaatggattgcgcgcctgcgccatattaaacgcctgcgccagcgcatttgcgaagaagcggcgtatagcaacccgagcctgccgctggtgcatccgccgagccatagcaaagcgccggcgcagaccccggcggaaccgaccccgggctatgaagtgggccagcgcaaacgcctgattagcagcgtggaagattttaccgaatttgtgtaggcggccgcctcgag

SEQ ID NO: 21 - DNA, 3402 bp, Artificial Sequence
  synthetic construct
atggcaaaacctacatcaaaagatagcggactgaaagagaagttcaaaatcctgctgggactggggacaccacgccctaatccacggtccgccgagggcaagcagaccgagttcatcatcacagccgagatcctgcgcgagctgtctatggagtgcggcctgaacaatcggatcagaatgatcggccagatttgcgaggtggccaagaccaagaagtttgaggagcacgcagtggaggccctgtggaaggcagtggccgatctgctgcagcctgagagaccactggaggcaaggcacgccgtgctggccctgctgaaggccatcgtgcagggacagggagagcgcctgggcgtgctgcgggccctgttctttaaagtgatcaaggactaccctagcaacgaggatctgcacgagagactggaggtgttcaaggccctgaccgataatggcaggcacatcacatatctggaggaggagctggccgactttgtgctgcagtggatggatgtgggcctgagctccgagttcctgctggtgctggtgaacctggtgaagtttaattcctgctacctggacgagtatatcgcccgcatggtgcagatgatctgcctgctgtgcgtgcggaccgcctctagcgtggacatcgaggtgtctctgcaggtgctggatgccgtggtgtgctacaactgtctgcccgccgagagcctgcctctgttcatcgtgaccctgtgcagaacaatcaatgtgaaggagctgtgcgagccttgttggaagctgatgaggaacctgctgggcacacacctgggacacagcgccatctacaatatgtgccacctgatggaggaccgcgcctatatggaggatgcaccactgctgaggggagccgtgttctttgtgggaatggcactgtggggagcacacagactgtactccctgaggaactctccaaccagcgtgctgccctctttttaccaggctatggcctgtccaaatgaggtggtgtcttatgagatcgtgctgagcatcacacgcctgatcaagaagtatcggaaggagctgcaggtggtggcctgggacatcctgctgaacatcatcgagcgcctgctgcagcagctgcagaccctggacagcccagagctgaggacaatcgtgcacgatctgctgaccacagtggaggagctgtgcgaccagaatgagttccacggctcccaggagcggtactttgagctggtggagcggtgcgcagatcagaggccagagtcctctctgctgaacctgatctcctatcgggcccagtctatccaccctgccaaggacggctggattcagaatctgcaggccctgatggagagcggaggaggctccggaggaggctctggaggcggcagcggcggcggcggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagtggcctctttctcctccctgtaccagtccagctgccaaggacagctgcacaggagcgtttcctgggcagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtg

SEQ ID NO: 22 - DNA, 3306 bp, Artificial Sequence
  synthetic construct
atggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggagcggaggaggctccggaggaggctctggaggcggcagcggcggcggcggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagtggcctctttctcctccctgtaccagtccagctgccaaggacagctgcacaggagcgtttcctgggcagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtg

SEQ ID NO: 23 - DNA, 3927 bp, Artificial Sequence
  synthetic construct
atggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggagcggaggaggctccggaggaggctctggaggcggcagcggcggcggcttcagggcccggagtactagtctcaacgagagacccaagagtctgaggatagccagaccccccaaacaaggcttgaataactctccacccgtgaaagaattcaaggagagctctgcagccgaggccttccggtgccgcagcatcagtgtgtctgaacatgtggtccgcagcaggatacagacgtccctcaccagtgccagcttggggtctgcagatgagaactccgtggcccaggctgacgatagcctgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagtggcctctttctcctccctgtaccagtccagctgccaaggacagctgcacaggagcgtttcctgggcagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtg

SEQ ID NO: 24 - DNA, 3801 bp, Artificial Sequence
  synthetic construct
atggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggagcggaggaggctccggaggaggctctggaggcggcagcggcggcggcttcagggcccggagtactagtctcaacgagagacccaagagtagcaggatacagacgtccctcaccagtgccagcttggggtctgcagatgagaactccgtggcccaggctgacgatagcctgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacagggaacaccagctggctgatgagcctggagaacccgctcagccctttctcctcggacatcaacaacatgcccctgcaggagctgtctaacgccctcatggcggctgagcgcttcaaggagcaccgggacacagccctgtacaagtcactgtcggtgccggcagccagcacggccaaaccccctcctctgcctcgctccaacacagtggcctctttctcctccctgtaccagtccagctgccaaggacagctgcacaggagcgtttcctgggcagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtg

SEQ ID NO: 25 - DNA, 3600 bp, Artificial Sequence
  synthetic construct
atggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggagcggaggaggctccggaggaggctctggaggcggcagcggcggcggcttcagggcccggagtactagtctcaacgagagacccaagagtagcaggatacagacgtccctcaccagtgccagcttggggtctgcagatgagaactccgtggcccaggctgacgatagcctgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacaggggtggcctctttctcctccctgtaccagtccagctgccaaggacagctgcacaggagcgtttcctgggcagactccgccgtggtcatggaggagggaagtccgggcgaggttcctgtgctggtggagcccccagggttggaggacgttgaggcagcgctaggcatggacaggcgcacggatgcctacagcaggtcgtcctcagtctccagccaggaggagaagtcgctccacgcggaggagctggttggcaggggcatccccatcgagcgagtcgtctcctcggagggtggccggccctctgtggacctctccttccagccctcgcagcccctgagcaagtccagctcctctcccgagctgcagactctgcaggacatcctcggggaccctggggacaaggccgacgtgggccggctgagccctgaggttaaggcccggtcacagtcagggaccctggacggggaaagtgctgcctggtcggcctcgggcgaagacagtcggggccagcccgagggtcccttgccttccagctccccccgctcgcccagtggcctccggccccgaggttacaccatctccgactcggccccatcacgcaggggcaagagagtagagagggacgccttaaagagcagagccacagcctccaatgcagagaaagtgccaggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtg

SEQ ID NO: 26 - DNA, 3114 bp, Artificial Sequence
  synthetic construct
atggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggagcggaggaggctccggaggaggctctggaggcggcagcggcggcggcttcagggcccggagtactagtctcaacgagagacccaagagtagcaggatacagacgtccctcaccagtgccagcttggggtctgcagatgagaactccgtggcccaggctgacgatagcctgaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcctggactcgggggagctgcagtccggcccggagtcgagctccagccccggggtgcatgtgagacagaccaaggaggcgccggccaagctggagtcccaggctgggcagcaggtgtcccgtggggcccgggatcgggtccgttccatgtcggggggccatggtcttcgagttggcgccctggacgtgccggcctcccagttcctgggcagtgccacttctccaggaccacggactgcaccagccgcgaaacctgagaaggcctcagctggcacccgggttcctgtgcaggagaagacgaacctggcggcctatgtgcccctgctgacccagggctgggcggagatcctggtccggaggcccacaggggtggcctctttctcctccctgtaccagtccagctgccaaggacagctgcacaggagcgtttcctgggcagactccgccgtggtcatggaggagggaagtccgggcgaggttcctgtgctggtggagcccccagggttggaggacgttgaggcagcgctaggcatggacaggcgcacggatgcctacagcaggagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagcctacctctggtgcaccctccgtcccatagcaaagcccctgcacagactccagccgagcccacacctggctatgaggtgggccagcggaagcgcctcatctcctcggtggaggacttcaccgagtttgtg

SEQ ID NO: 27 - DNA, 130 bp, Unknown
  5′ ITR
ctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 28 - DNA, 130 bp, Unknown
  3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 29 - protein, 42 aa, Homo sapiens
LRIARPPKQGLNNSPPVKEFKESSAAEAFRCRSISVSEHVVR

SEQ ID NO: 30 - protein, 67 aa, Homo sapiens
NTSWLMSLENPLSPFSSDINNMPLQELSNALMAAERFKEHRDTALYKSLSVPAASTAKPPPLPRSNT

SEQ ID NO: 31 - protein, 162 aa, Homo sapiens
SSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINP

SEQ ID NO: 32 - protein, 177 aa, Homo sapiens
FRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAP

SEQ ID NO: 33 - DNA, 382 bp, Unknown
  CMV-IE URE
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 34 - DNA, 260 bp, Unknown
  CB6 promoter
ccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 35 - DNA, 6 bp, Unknown
  Kozak sequence
gccacc

SEQ ID NO: 36 - DNA, 56 bp, Unknown
  Rabbit polyA sequence
aataaaggaaatttattttcattgcaatagtgtgttggaattttttgtgtctctca

(19) United States
(12) Patent Application Publication
(10) Pub. No.: US 2024/0261439 A1
(43) Pub. Date: Aug. 8, 2024

(54) GENE THERAPY FOR TUBEROUS SCLEROSIS

(71) Applicant: BridgeBio Gene Therapy Research, Inc., Raleigh, NC (US)

(72) Inventor: David Wayne SCOTT, Cary, NC (US)

(21) Appl. No.: 18/570,040

(22) PCT Filed: Jun. 14, 2022
(86) PCT No.: PCT/US2022/033452

§ 371 Date: Dec. 13, 2023

Related U.S. Application Data
(60) Provisional application No. 63/210,456, filed on Jun. 14, 2021.

(51) Int. Cl. A61K 48/00; A61K 31/436; A61K 38/00; C07K 14/47; C12N 7/00; C12N 15/86

(52) U.S. Cl. CPC A61K 48/0058; A61K 31/436; C07K 14/4706; C12N 7/00; C12N 15/86; A61K 38/00; C12N 2750/14143; C12N 2750/14151

(57) ABSTRACT

The disclosure provides gene therapy compositions and methods for treating tuberous sclerosis. In particular, the disclosure provides compositions comprising recombinant adeno-associated viruses (rAAVs) comprising an AAV capsid protein, and an AAV expression cassette encoding condensed Tuberins (cTuberins), and methods of use thereof.

### CROSS REFERENCE TO RELATED APPLICATIONS

This application claims the benefit of priority to U.S. Provisional Application No. 63/210,456, filed on Jun. 14, 2021, the contents of which are hereby incorporated by reference in their entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: BGTR_003_01 WO_SeqList_ST25.txt, date created: Jun. 14, 2022, file size ˜170,338 bytes).

### BACKGROUND

Tuberous sclerosis complex (TSC), also referred to as tuberous sclerosis, is a multisystem, autosomal dominant, genetic disease that can result in non-cancerous tumors growing in the brain and in other vital organs such as the kidneys, heart, liver, eyes, lungs and skin. In the brain, such tumors can cause developmental delay, autism, epilepsy, and hydrocephalus. Life-threatening conditions associated with tuberous sclerosis include renal angiomyolipomas, which can cause internal bleeding, and lymphangioleiomyomatosis (LAM), which can compromise breathing.

Tuberous sclerosis is caused by one or more mutations in the TSC1 gene and/or the TSC2 gene. Tuberous sclerosis resulting from mutations in TSC2 is more severe and more prevalent. TSC1 and TSC2 are tumor growth suppressor genes that code for the proteins hamartin and tuberin, respectively. Tuberin and hamartin form a protein complex that integrates multiple signals to regulate mammalian target of rapamycin (mTOR) signaling, primarily by inhibiting the mTORC1 complex. In addition, tuberin also contains a GTPase activating domain (GAP) domain, which downregulates the mTORC1 activator, Rheb.

Current treatment of tuberous sclerosis involves administration of rapamycin and its analogues. However, these drugs must be administered continuously and have notable side effects, including compromised brain development and immune suppression. Administration of rapamycin and its analogues may also give rise to adverse events due to the over-suppression of mTORC1 activity. In addition, some patients do not respond to rapamycin, or respond initially and then become resistant.

Accordingly, there is an unmet need for compositions and methods that can be used to treat tuberous sclerosis, particularly compositions and methods based on gene therapy.

### SUMMARY

The present disclosure provides compositions and methods for use in the treatment of tuberous sclerosis complex. In some embodiments, the present disclosure provides condensed tuberin proteins (cTuberins) comprising (i) an N-terminal region capable of binding hamartin, and (ii) a C-terminal GTPase-activating protein (GAP) region, wherein the cTuberin lacks certain amino acid residues of human tuberin (SEQ ID NO: 1). In some embodiments, a cTuberin of the present disclosure lacks amino acid residues 451 to 932 of human tuberin (SEQ ID NO: 1). In some embodiments, a cTuberin of the present disclosure lacks amino acid residues 419 to 932 of human tuberin (SEQ ID NO: 1). In some embodiments, a cTuberin of the present disclosure comprises (i) an N-terminal region capable of binding hamartin, and (ii) a C-terminal GTPase-activating protein (GAP) region, wherein the C-terminal region comprises an amino acid sequence with at least 90% identity to SEQ ID NO: 7, and lacks amino acid residues 451 to 932 of SEQ ID NO: 1. In some embodiments, a cTuberin of the present disclosure comprises (i) an N-terminal region capable of binding hamartin, and (ii) a C-terminal GTPase-activating protein (GAP) region, wherein the C-terminal region comprises an amino acid sequence with at least 90% identity to one of SEQ ID NOs: 10-12, and wherein the cTuberin lacks amino acid residues 451 to 932 of SEQ ID NO: 1.

In addition to cTuberin proteins, the present disclosure provides nucleic acid molecules encoding any one of the cTuberin proteins disclosed herein as well as compositions configured to cause expression of any one of the cTuberin proteins disclosed herein in a given cell. For example, the present disclosure provides adeno-associated virus (AAV) expression cassettes comprising, from 5′ to 3′: a 5′ AAV inverted terminal repeat (ITR); any one of the nucleic acid molecules disclosed herein; and a 3′ AAV ITR. The present disclosure also provides recombinant AAVs (rAAVs), comprising: an AAV capsid protein, and any one of the nucleic acid molecules or AAV expression cassettes disclosed herein. Moreover, the present disclosure provides compositions including pharmaceutical compositions comprising any one of the cTuberin proteins, any one of the nucleic acid molecules, any one of the plasmids, any one of the host cells, or any one of the rAAVs disclosed herein.

In another aspect, the present disclosure provides methods of expressing any one of the cTuberins disclosed herein in a target cell, comprising: contacting any one of the nucleic acid molecules disclosed herein, any one of the plasmids disclosed herein, any one of the rAAVs disclosed herein, any one of the extracellular vesicles (EVs) disclosed herein, or any one of the compositions disclosed herein with the target cell, thereby expressing the cTuberin in the target cell.

In a further aspect, the present disclosure provides methods of treating tuberous sclerosis in a subject in need thereof, comprising: administering to the subject a therapeutically effective amount of any one of the nucleic acid molecules disclosed herein, any one of the plasmids disclosed herein, any one of the rAAVs disclosed herein, any one of the EVs disclosed herein, or any one of the compositions disclosed herein, thereby treating tuberous sclerosis in the subject.

### DETAILED DESCRIPTION

Using AAV-based gene therapy to treat tuberous sclerosis or TSC caused by mutations in TSC2 has been complicated by the relatively small insert capacity of an AAV vector (˜4.7 kb) compared to the 5.4 kb cDNA of human tuberin. The present disclosure provides condensed forms of the human tuberin gene (TSC2) that are small enough to be incorporated in an AAV vector and encode condensed tuberins (cTuberins). In particular, the present disclosure provides compositions comprising recombinant adeno-associated viruses (rAAVs) comprising an AAV capsid protein, and an AAV expression cassette encoding a cTuberin, and methods of use thereof, including in the treatment of tuberous sclerosis.

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

The terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 0% to 10%.

Also as used herein, “and/or” refers to and encompasses any and all possible combinations of one or more of the associated listed items, as well as the lack of combinations when interpreted in the alternative (“or”). The use of the alternative (e.g., “or”) should be understood to mean either one, both, or any combination thereof of the alternatives.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

As used herein, “treatment,” “treating,” “palliating.” and “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition: (2) inhibiting the state, disorder or condition (e.g., arresting, reducing or delaying the development of the disease, or a relapse thereof in case of maintenance treatment, of at least one clinical or subclinical symptom thereof); and (3) relieving the condition (for example, by causing regression, or reducing the severity of the state, disorder or condition or at least one of its clinical or subclinical symptoms). For example, beneficial clinical results include, but are not limited to, delay or slowing of invasiveness or growth of tumors or hamartomas, and amelioration of symptoms associated with such tumors or hamartomas. For example, in the case of renal angiomyolipomas, tumor size can be monitored by magnetic resonance imaging (MRI) and the shrinkage in cell size due to the administration of any one of the compositions disclosed herein can be analyzed according to standard procedures, such as those used to monitor treatment of tuberous sclerosis using rapamycin. Treatment also includes a decrease in mortality or an increase in the lifespan of a subject as compared to one not receiving the treatment.

The term “effective amount” or “therapeutically effective amount” refers to the amount of an agent that is sufficient to achieve an outcome, for example, to effect beneficial or desired results, such as, treatment of tuberous sclerosis or a symptom thereof. The therapeutically effective amount may vary depending upon one or more of: the subject and disease condition being treated, the weight and age of the subject, the severity of the disease condition, the manner of administration, and the like. A therapeutically effective amount may be an amount sufficient to treat tuberous sclerosis and/or to ameliorate, diminish the severity of, eliminate, and/or delay the onset of one or more symptoms of tuberous sclerosis. In some embodiments, a therapeutically effective amount may be an amount sufficient to express a tuberin (e.g., a tuberin lacking one or more mutations, such as a condensed tuberin provided herein) in a subject.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. In some embodiments, the subject is a human. A human subject may be an adult, a teenager (e.g., 12 years to 18 years of age), a child (e.g., 2 years to 14 years of age), an infant (e.g., 1 month to 24 months old), or a neonate (up to 1 month old). In some embodiments, an adult is a senior about 60 years or older, such as about 65 years or older. In some embodiments, the subject is a pregnant woman or a woman intending to become pregnant. In some embodiments, the subject is less than 18 years of age.

An “adeno-associated virus (AAV) expression cassette” is a nucleic acid that gets packaged into a recombinant AAV vector, and comprises a sequence encoding one or more transgenes flanked by a 5′ inverted terminal repeat (ITR) and a 3′ITR.

As used herein, the terms “virus vector,” “viral vector,” and “gene delivery vector” refer to a virus particle that functions as a nucleic acid delivery vehicle, and which comprises a nucleic acid molecule (e.g., an AAV expression cassette) packaged within a virion. Exemplary virus vectors include adeno-associated virus vectors (AAVs).

As used herein, the term “adeno-associated virus” (AAV), includes but is not limited to, AAV type 1 (e.g., AAV of serotype 1, also referred to as AAV1), AAV type 2 (e.g., AAV2), AAV type 3 (e.g., AAV3, including types 3A and 3B, AAV3A and AAV3B), AAV type 4 (e.g., AAV4), AAV type 5 (e.g., AAV5), AAV type 6 (e.g., AAV6), AAV type 7 (e.g., AAV7), AAV type 8 (e.g., AAV8), AAV type 9 (e.g., AAV9), AAV type 10 (e.g., AAV10), AAV type 11 (e.g., AAV11), AAV type 12 (e.g., AAV12), AAV type 13 (e.g., AAV13), AAV type rh32.33 (e.g., AAVrh32.33), AAV type rh8 (e.g., AAVrh8), AAV type rh10) (e.g., AAVrh10), AAV type rh74 (e.g., AAVrh74), AAV type hu.68 (e.g., AAVhu.68), avian AAV (e.g., AAAV), bovine AAV (e.g., BAAV), canine AAV, equine AAV, ovine AAV, snake AAV, bearded dragon AAV, AAV218, AAV2g9, AAV-LK03, AAV7m8, AAV Anc80, AAV PHP.B. and any other AAV now known or later discovered.

As used herein “sequence identity” refers to the extent to which two optimally aligned polynucleotides or polypeptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. “Percent identity” is the identity fraction times 100. The extent of identity (homology) between two sequences can be ascertained using a computer program and mathematical algorithm. Percentage identity can be calculated using the alignment program Clustal Omega, available at www.ebi.ac.uk/Tools/msa/clustalo using default parameters. See, Sievers et al., “Fast, scalable generation of high-quality protein multiple sequence alignments using Clustal Omega.” (2011 Oct. 11) Molecular systems biology 7:539. For the purposes of calculating identity to a sequence, extensions such as tags are not included.

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein.

As used herein, “codon optimization” refers to modifying a nucleic acid sequence to change individual nucleic acids without any resulting change in the corresponding encoded amino acid. Sequences modified in this way are referred to herein as “codon optimized.” Methods of performing codon optimization are described in U.S. Pat. Nos. 7,561,972, 7,561,973, and 7,888,112, each of which is incorporated herein by reference in their entireties for all purposes. In some embodiments, the sequence surrounding the translational start site can be converted to a consensus Kozak sequence as described further in Kozak et al., Nucleic Acids Res. 15(20):8125-81 48 (1987), incorporated herein by reference in its entirety for all purposes.

Condensed Tuberin (cTuberin)

As used herein, a condensed tuberin or cTuberin refers to a recombinant tuberin protein that has a deletion of one or more amino acid residues, as compared to the native tuberin protein sequence. In some embodiments, the native tuberin is a human tuberin. In some embodiments, the native tuberin protein sequence has the amino acid sequence of SEQ ID NO: 1, which sequence includes 1807 amino acid residues. In some embodiments, a cTuberin provided herein lacks at least one amino acid residue of SEQ ID NO: 1. In some embodiments, a cTuberin provided herein lacks at least one amino residue of SEQ ID NO: 1 from the region between the N-terminal and C-terminal regions.

In some embodiments, the amino acid and nucleic acid sequences of human tuberin are found at NCBI Accession No. NP_000539.2 and GenBank Accession No. X75621.1, respectively. In some embodiments, the amino acid sequences of human tuberin include, but are not limited to, tuberin isoform 4 (NCBI Accession No. NP_001070651.1), tuberin isoform 5 (NCBI Accession No. NP_001107854.1), tuberin isoform 6 (NCBI Accession No. NP_001305756.1), tuberin isoform 7 (NCBI Accession No. NP_001305758.1), tuberin isoform 8 (NCBI Accession No. NP_001305760.1), tuberin isoform 9 (NCBI Accession No. NP_001305761.1), tuberin isoform X7 (NCBI Accession No. XP_024306181.1), tuberin isoform X8 (NCBI Accession No. XP_005255586.2), tuberin isoform X9 (NCBI Accession No. XP_016879105.1), tuberin isoform X10 (NCBI Accession No. XP_005255588.2), tuberin isoform X11 (NCBI Accession No. XP_016879106.1), tuberin isoform X12 (NCBI Accession No. XP_016879107.1), and others.

In some embodiments, the cTuberin comprises an N-terminal region capable of binding hamartin. In some embodiments, the cTuberin comprises a C-terminal GTPase-activating protein (GAP) region. In some embodiments, the one or more amino acid residues that are deleted in the cTuberin lie between the N-terminal region capable of binding hamartin, and the C-terminal GTPase-activating protein (GAP) region.

In some embodiments, the cTuberin comprises, or consists of, an N-terminal region capable of binding hamartin, and a C-terminal GTPase-activating protein (GAP) region. In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as about 100%) identity to SEQ ID NO: 4 or 5. In some embodiments, the N-terminal region comprises, or consists of, an amino acid sequence with at least about 90% identity to SEQ ID NO: 4 or 5. In some embodiments, the N-terminal region comprises, or consists of, the amino acid sequence of SEQ ID NO: 4 or 5.

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to any one of SEQ ID NOs: 7-12. In some embodiments, the C-terminal region comprises, or consists of, an amino acid sequence with at least about 90% identity to any one of SEQ ID NOs: 7-12. In some embodiments, the C-terminal region comprises, or consists of, an amino acid sequence of any one of SEQ ID NOs: 7-12.

In some embodiments, the N-terminal region comprises, or consists of, an amino acid sequence with at least about 75% (for example, about 80%, about 85%, about 90%, about 95%, about 96%, about 97%, about 98%, about 99%, or 100%) identity to SEQ ID NO: 4; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 7. In some embodiments, the N-terminal region comprises, or consists of, an amino acid sequence with at least about 90% identity to SEQ ID NO: 4; and the C-terminal region comprises, or consists of, an amino acid sequence with at least about 90% identity to SEQ ID NO: 7. In some embodiments, the N-terminal region comprises, or consists of, the amino acid sequence of SEQ ID NO: 4; and the C-terminal region comprises, or consists of, the amino acid sequence of SEQ ID NO: 7.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 5; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 7. In some embodiments, the N-terminal region comprises, or consists of, an amino acid sequence with at least about 90% identity to SEQ ID NO: 5; and the C-terminal region comprises, or consists of, an amino acid sequence with at least about 90% identity to SEQ ID NO: 7. In some embodiments, the N-terminal region comprises, or consists of, the amino acid sequence of SEQ ID NO: 5; and the C-terminal region comprises, or consists of, the amino acid sequence of SEQ ID NO: 7.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 4; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 8. In some embodiments, the N-terminal region comprises, or consists of, an amino acid sequence with at least about 90% identity to SEQ ID NO: 4; and the C-terminal region comprises, or consists of, an amino acid sequence with at least about 90% identity to SEQ ID NO: 8. In some embodiments, the N-terminal region comprises, or consists of, the amino acid sequence of SEQ ID NO: 4; and the C-terminal region comprises, or consists of, the amino acid sequence of SEQ ID NO: 8.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 5; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 8. In some embodiments, the N-terminal region comprises, or consists of, an amino acid sequence with at least about 90% identity to SEQ ID NO: 5; and the C-terminal region comprises, or consists of, an amino acid sequence with at least about 90% identity to SEQ ID NO: 8. In some embodiments, the N-terminal region comprises, or consists of, the amino acid sequence of SEQ ID NO: 5; and the C-terminal region comprises, or consists of, the amino acid sequence of SEQ ID NO: 8.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 4; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 9. In some embodiments, the N-terminal region comprises, or consists of, an amino acid sequence with at least about 90% identity to SEQ ID NO: 4; and the C-terminal region comprises, or consists of, an amino acid sequence with at least about 90% identity to SEQ ID NO: 9. In some embodiments, the N-terminal region comprises, or consists of, the amino acid sequence of SEQ ID NO: 4; and the C-terminal region comprises, or consists of, the amino acid sequence of SEQ ID NO: 9.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 5; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 9. In some embodiments, the N-terminal region comprises, or consists of, an amino acid sequence with at least about 90% identity to SEQ ID NO: 5; and the C-terminal region comprises, or consists of, an amino acid sequence with at least about 90% identity to SEQ ID NO: 9. In some embodiments, the N-terminal region comprises, or consists of, the amino acid sequence of SEQ ID NO: 5; and the C-terminal region comprises, or consists of, the amino acid sequence of SEQ ID NO: 9.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 4; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 10. In some embodiments, the N-terminal region comprises, or consists of, an amino acid sequence with at least about 90% identity to SEQ ID NO: 4; and the C-terminal region comprises, or consists of, an amino acid sequence with at least about 90% identity to SEQ ID NO: 10. In some embodiments, the N-terminal region comprises, or consists of, the amino acid sequence of SEQ ID NO: 4; and the C-terminal region comprises, or consists of, the amino acid sequence of SEQ ID NO: 10.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 5; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 10. In some embodiments, the N-terminal region comprises, or consists of, an amino acid sequence with at least about 90% identity to SEQ ID NO: 5; and the C-terminal region comprises, or consists of, an amino acid sequence with at least about 90% identity to SEQ ID NO: 10. In some embodiments, the N-terminal region comprises, or consists of, the amino acid sequence of SEQ ID NO: 5; and the C-terminal region comprises, or consists of, the amino acid sequence of SEQ ID NO: 10.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 4; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 11. In some embodiments, the N-terminal region comprises, or consists of, an amino acid sequence with at least about 90% identity to SEQ ID NO: 4; and the C-terminal region comprises, or consists of, an amino acid sequence with at least about 90% identity to SEQ ID NO: 11. In some embodiments, the N-terminal region comprises, or consists of, the amino acid sequence of SEQ ID NO: 4; and the C-terminal region comprises, or consists of, the amino acid sequence of SEQ ID NO: 11.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 5; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 11. In some embodiments, the N-terminal region comprises, or consists of, an amino acid sequence with at least about 90% identity to SEQ ID NO: 5; and the C-terminal region comprises, or consists of, an amino acid sequence with at least about 90% identity to SEQ ID NO: 11. In some embodiments, the N-terminal region comprises, or consists of, the amino acid sequence of SEQ ID NO: 5; and the C-terminal region comprises, or consists of, the amino acid sequence of SEQ ID NO: 11.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 4; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 12. In some embodiments, the N-terminal region comprises, or consists of, an amino acid sequence with at least about 90% identity to SEQ ID NO: 4; and the C-terminal region comprises, or consists of, an amino acid sequence with at least about 90% identity to SEQ ID NO: 12. In some embodiments, the N-terminal region comprises, or consists of, the amino acid sequence of SEQ ID NO: 4; and the C-terminal region comprises, or consists of, the amino acid sequence of SEQ ID NO: 12.

In some embodiments, the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 5; and the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 12. In some embodiments, the N-terminal region comprises, or consists of, an amino acid sequence with at least about 90% identity to SEQ ID NO: 5; and the C-terminal region comprises, or consists of, an amino acid sequence with at least about 90% identity to SEQ ID NO: 12. In some embodiments, the N-terminal region comprises, or consists of, the amino acid sequence of SEQ ID NO: 5; and the C-terminal region comprises, or consists of, the amino acid sequence of SEQ ID NO: 12.

The present disclosure provides cTuberins comprising or consisting of (i) an N-terminal region capable of binding hamartin, and (ii) a C-terminal GTPase-activating protein (GAP) region, wherein the cTuberin lacks amino acid residues 451-932 of SEQ ID NO: 1. The present disclosure also provides cTuberins comprising or consisting of (i) an N-terminal region capable of binding hamartin, and (ii) a C-terminal GTPase-activating protein (GAP) region, wherein the cTuberin lacks amino acid residues 419-932 of SEQ ID NO: 1. Where amino acid ranges are denoted, the ranges are inclusive (e.g., a cTuberin lacking amino acid residues “419-932” or “419 to 932” means the cTuberin lacks amino acids 419 and 932 of SEQ ID NO: 1 as well as all amino acid residues disposed between them).

In some embodiments, the cTuberin further lacks amino acid residues 947-988 of SEQ ID NO: 1. In some embodiments, the cTuberin lacks amino acid residues 451-932 and 947-988 of SEQ ID NO: 1. In some embodiments, the cTuberin lacks amino acid residues 419 to 932 and amino acid residues 947-988 of SEQ ID NO: 1.

In some embodiments, the cTuberin further lacks amino acid residues 1205-1271 of SEQ ID NO: 1. In some embodiments, the cTuberin lacks amino acid residues 451-932 and 1205-1271 of SEQ ID NO: 1. In some embodiments, the cTuberin lacks amino acid residues 419 to 932 and 1205-1271 of SEQ ID NO: 1.

In some embodiments, the cTuberin lacks amino acid residues 451-932, 947-988, and 1205-1271 of SEQ ID NO: 1. In some embodiments, the cTuberin lacks amino acid residues 419-932, 947-988, and 1205-1271 of SEQ ID NO: 1.

In some embodiments, the cTuberin further lacks amino acid residues 1336-1497 of SEQ ID NO: 1. In some embodiments, the cTuberin lacks amino acid residues 419-932 and 1336-1497 of SEQ ID NO: 1. In some embodiments, the cTuberin lacks amino acid residues 451-932 and 1336-1497 of SEQ ID NO: 1. In some embodiments, the cTuberin lacks amino acid residues 419-932, 947-988, 1205-1271, and 1336-1497 of SEQ ID NO: 1. In some embodiments, the cTuberin lacks amino acid residues 451-932, 947-988, 1205-1271, and 1336-1497 of SEQ ID NO: 1.

In some embodiments, the cTuberin lacks amino acid residues 419-932, 1205-1271, and 1336-1497 of SEQ ID NO: 1. In some embodiments, the cTuberin lacks amino acid residues 451-932, 1205-1271, and 1336-1497 of SEQ ID NO: 1. In some embodiments, the cTuberin lacks amino acid residues 419-932, 947-988, and 1336-1497 of SEQ ID NO: 1. In some embodiments, the cTuberin lacks amino acid residues 451-932, 947-988, and 1336-1497 of SEQ ID NO: 1.

In some embodiments, the cTuberin further lacks amino acid residues 933-1109 of human tuberin (SEQ ID NO: 1). In some embodiments, the cTuberin lacks amino acid residues 451-932 and 933-1109 of human tuberin (SEQ ID NO: 1). In some embodiments, the cTuberin lacks amino acid residues 419-932 and 933-1109 of human tuberin (SEQ ID NO: 1). In some embodiments, the cTuberin lacks amino acid residues 419-1109 of SEQ ID NO: 1.

In some embodiments, the cTuberin lacks amino acid residues 451-932 of human tuberin (SEQ ID NO: 1). In some embodiments, the cTuberin lacks amino acid residues 451-1109 of human tuberin (SEQ ID NO: 1). In some embodiments, the cTuberin lacks amino acid residues 451-1139 of human tuberin (SEQ ID NO: 1). In some embodiments, the cTuberin lacks amino acid residues 451-1514 of human tuberin (SEQ ID NO: 1). In some embodiments, the cTuberin lacks amino acid residues 419-932 of human tuberin. In some embodiments, the cTuberin lacks amino acid residues 419-1109 of human tuberin (SEQ ID NO: 1). In some embodiments, the cTuberin lacks amino acid residues 419-1139 of human tuberin (SEQ ID NO: 1). In some embodiments, the cTuberin lacks amino acid residues 419-1514 of human tuberin (SEQ ID NO: 1).

In some embodiments, the cTuberin lacks amino acid residues of one or more exons of human tuberin (SEQ ID NO:1), such as exon 25, 30, and/or 33. In some embodiments, the cTuberin lacks the amino acid residues of exon 25 of human tuberin (SEQ ID NO: 1). In some embodiments, the cTuberin lacks the amino acid residues of exon 30 of human tuberin (SEQ ID NO: 1). In some embodiments, the cTuberin lacks the amino acid residues of exon 33 of human tuberin (SEQ ID NO: 1). In some embodiments, the cTuberin lacks the amino acid residues of exons 25 and 30 of human tuberin (SEQ ID NO: 1). In some embodiments, the cTuberin lacks the amino acid residues of exons 25, 30, and 33 of human tuberin (SEQ ID NO: 1). In some embodiments, the cTuberin lacks amino acid residues 451-932 and the amino acid residues of exon 25 of human tuberin (SEQ ID NO: 1). In some embodiments, the cTuberin lacks amino acid residues 451-932 and the amino acid residues of exons 25 and 30 of human tuberin (SEQ ID NO: 1). In some embodiments, the cTuberin lacks amino acid residues 451-932 and the amino acid residues of exons 25, 30, and 33 of human tuberin (SEQ ID NO: 1). In some embodiments, the cTuberin lacks amino acid residues 419-932 and the amino acid residues of exon 25 of human tuberin (SEQ ID NO: 1). In some embodiments, the cTuberin lacks amino acid residues 419-932 and the amino acid residues of exons 25 and 30 of human tuberin (SEQ ID NO: 1). In some embodiments, the cTuberin lacks amino acid residues 419-932 and the amino acid residues of exons 25, 30, and 33 of human tuberin (SEQ ID NO: 1).

The present disclosure provides condensed tuberins (cTuberins) comprising or consisting of (i) an N-terminal region capable of binding hamartin, and (ii) a C-terminal GTPase-activating protein (GAP) region, wherein the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 7, and wherein the cTuberin lacks amino acid residues 419-932 or 451-932 of human tuberin (SEQ ID NO: 1). The present disclosure also provides condensed tuberins (cTuberins) comprising or consisting of (i) an N-terminal region capable of binding hamartin, and (ii) a C-terminal GTPase-activating protein (GAP) region, wherein the C-terminal region comprises, or consists of, an amino acid sequence with at least about 90% identity to SEQ ID NO: 7, and wherein the cTuberin lacks amino acid residues 419-932 or 451-932 of human tuberin (SEQ ID NO: 1). The present disclosure further provides condensed tuberins (cTuberins) comprising or consisting of (i) an N-terminal region capable of binding hamartin, and (ii) a C-terminal GTPase-activating protein (GAP) region, wherein the C-terminal region comprises, or consists of, the amino acid sequence of SEQ ID NO: 7, and wherein the cTuberin lacks amino acid residues 419-932 or 451-932 of human tuberin (SEQ ID NO: 1).

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 8 and the cTuberin lacks amino acid residues 419-932 or 451-932 of human tuberin (SEQ ID NO: 1). In some embodiments, the C-terminal region comprises, or consists of, an amino acid sequence with at least about 90% identity to SEQ ID NO: 8 and the cTuberin lacks amino acid residues 419-932 or 451-932 of human tuberin (SEQ ID NO: 1). In some embodiments, the C-terminal region comprises, or consists of, the amino acid sequence of SEQ ID NO: 8 and the cTuberin lacks amino acid residues 419-932 or 451-932 of human tuberin (SEQ ID NO: 1).

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 9 and the cTuberin lacks amino acid residues 419-932 or 451-932 of human tuberin (SEQ ID NO: 1). In some embodiments, the C-terminal region comprises, or consists of, an amino acid sequence with at least about 90% identity to SEQ ID NO: 9 and the cTuberin lacks amino acid residues 419-932 or 451-932 of human tuberin (SEQ ID NO: 1). In some embodiments, the C-terminal region comprises, or consists of, the amino acid sequence of SEQ ID NO: 9 and the cTuberin lacks amino acid residues 419-932 or 451-932 of human tuberin (SEQ ID NO: 1).

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 7, the cTuberin lacks amino acid residues 451-932 of human tuberin (SEQ ID NO: 1), and the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 4. In some embodiments, the C-terminal region comprises, or consists of, an amino acid sequence with at least about 90% identity to SEQ ID NO: 7, the cTuberin lacks amino acid residues 451-932 of human tuberin (SEQ ID NO: 1), and the N-terminal region comprises, or consists of, an amino acid sequence with at least 90% identity to SEQ ID NO: 4. In some embodiments, the C-terminal region comprises, or consists of, the amino acid sequence of SEQ ID NO: 7, the cTuberin lacks amino acid residues 451-932 of human tuberin (SEQ ID NO: 1), and the N-terminal region comprises, or consists of, the amino acid sequence of SEQ ID NO: 4.

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 7, the cTuberin lacks amino acid residues 419-932 or 451-932 of human tuberin (SEQ ID NO: 1), and the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 5. In some embodiments, the C-terminal region comprises, or consists of, an amino acid sequence with at least about 90% identity to SEQ ID NO: 7, the cTuberin lacks amino acid residues 419-932 or 451-932 of human tuberin (SEQ ID NO: 1), and the N-terminal region comprises, or consists of, an amino acid sequence with at least 90% identity to SEQ ID NO: 5. In some embodiments, the C-terminal region comprises, or consists of, the amino acid sequence of SEQ ID NO: 7, the cTuberin lacks amino acid residues 419-932 or 451-932 of human tuberin (SEQ ID NO: 1), and the N-terminal region comprises, or consists of, the amino acid sequence of SEQ ID NO: 5.

In another aspect, the present disclosure provides condensed tuberins (cTuberins) comprising or consisting of (i) an N-terminal region capable of binding hamartin, and (ii) a C-terminal GTPase-activating protein (GAP) region, wherein the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to one of SEQ ID NOs: 10-12. The present disclosure provides condensed tuberins (cTuberins), comprising or consisting of (i) an N-terminal region capable of binding hamartin, and (ii) a C-terminal GTPase-activating protein (GAP) region, wherein the C-terminal region comprises, or consists of, an amino acid sequence with at least about 90% identity to one of SEQ ID NOs: 10-12. The present disclosure also provides condensed tuberins (cTuberins), comprising or consisting of (i) an N-terminal region capable of binding hamartin, and (ii) a C-terminal GTPase-activating protein (GAP) region, wherein the C-terminal region comprises, or consists of, the amino acid sequence of any one of SEQ ID NOs: 10-12.

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to one of SEQ ID NOs: 10-12 and the cTuberin lacks amino acid residues 419-932 or 451-932 of human tuberin (SEQ ID NO: 1). In some embodiments, the C-terminal region comprises, or consists of, an amino acid sequence with at least about 90% identity to one of SEQ ID NOS: 10-12 and the cTuberin lacks amino acid residues 419-932 or 451-932 of human tuberin (SEQ ID NO: 1). In some embodiments, the C-terminal region comprises, or consists of, the amino acid sequence of any one of SEQ ID NOs: 10-12 and the cTuberin lacks amino acid residues 419-932 or 451-932 of human tuberin (SEQ ID NO: 1).

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 10, and the cTuberin lacks amino acid residues 419-932 or 451-932 of human tuberin (SEQ ID NO: 1). In some embodiments, the C-terminal region comprises, or consists of, an amino acid sequence with at least about 90% identity to SEQ ID NO: 10, and the cTuberin lacks amino acid residues 419-932 or 451-932 of human tuberin (SEQ ID NO: 1). In some embodiments, the C-terminal region comprises, or consists of, the amino acid sequence of SEQ ID NO: 10, and the cTuberin lacks amino acid residues 419-932 or 451-932 of human tuberin (SEQ ID NO: 1).

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 11, and the cTuberin lacks amino acid residues 419-932 or 451-932 of human tuberin (SEQ ID NO: 1). In some embodiments, the C-terminal region comprises, or consists of, an amino acid sequence with at least about 90% identity to SEQ ID NO: 11, and the cTuberin lacks amino acid residues 419-932 or 451-932 of human tuberin (SEQ ID NO: 1). In some embodiments, the C-terminal region comprises, or consists of, the amino acid sequence of SEQ ID NO: 11, and the cTuberin lacks amino acid residues 419-932 or 451-932 of human tuberin (SEQ ID NO: 1).

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 12, and the cTuberin lacks amino acid residues 419-932 or 451-932 of human tuberin (SEQ ID NO: 1). In some embodiments, the C-terminal region comprises, or consists of, an amino acid sequence with at least about 90% identity to SEQ ID NO: 12, and the cTuberin lacks amino acid residues 419-932 or 451-932 of human tuberin (SEQ ID NO: 1). In some embodiments, the C-terminal region comprises, or consists of, the amino acid sequence of SEQ ID NO: 12, and the cTuberin lacks amino acid residues 419-932 or 451-932 of human tuberin (SEQ ID NO: 1).

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to one of SEQ ID NOs: 10-12 and the cTuberin lacks amino acid residues 419-932 or 451-932 of human tuberin (SEQ ID NO: 1), and the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 5. In some embodiments, the C-terminal region comprises, or consists of, an amino acid sequence with at least about 90% identity to one of SEQ ID NOs: 10-12 and the cTuberin lacks amino acid residues 419-932 or 451-932 of human tuberin (SEQ ID NO: 1), and the N-terminal region comprises, or consists of, an amino acid sequence with at least about 90% identity to SEQ ID NO: 5. In some embodiments, the C-terminal region comprises, or consists of, the amino acid sequence of any one of SEQ ID NOs: 10-12 and the cTuberin lacks amino acid residues 419-932 or 451-932 of human tuberin (SEQ ID NO: 1), and the N-terminal region comprises, or consists of, the amino acid sequence of SEQ ID NO: 5.

In some embodiments, the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to one of SEQ ID NOs: 10-12 and the cTuberin lacks amino acid residues 451-932 of human tuberin (SEQ ID NO: 1), and the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 4. In some embodiments, the C-terminal region comprises, or consists of, an amino acid sequence with at least about 90% identity to one of SEQ ID NOs: 10-12 and the cTuberin lacks amino acid residues 451-932 of human tuberin (SEQ ID NO: 1), and the N-terminal region comprises, or consists of, an amino acid sequence with at least about 90% identity to SEQ ID NO: 4. In some embodiments, the C-terminal region comprises, or consists of, the amino acid sequence of any one of SEQ ID NOs: 10-12 and the cTuberin lacks amino acid residues 451-932 of human tuberin (SEQ ID NO: 1), and the N-terminal region comprises, or consists of, the amino acid sequence of SEQ ID NO: 4.

In some embodiments, the cTuberin comprises a polypeptide spacer between the N-terminal region and the C-terminal region. In some embodiments, the polypeptide spacer comprises, or consists of, the sequence of SEQ ID NO: 2 (SGGG). In some embodiments, the polypeptide spacer comprises, or consists of, the sequence of SEQ ID NO: 3 (SGGGSGGG SGGGSGGG).

In some embodiments, the cTuberin comprises, or consists of, an amino acid sequence with at least about 75% (for example, at least about 80%, about 85%, about 90%, about 95%, about 96%, about 97%, about 98%, about 99%, or greater, such as 100%) identity to any one of SEQ ID NOs: 14-19. In some embodiments, the cTuberin comprises, or consists of, an amino acid sequence with at least about 90% identity to any one of SEQ ID NOs: 14-19. In some embodiments, the cTuberin comprises, or consists of, the amino acid sequence of any one of SEQ ID NOs: 14-19.

AAV Expression Cassettes Encoding cTuberin

In addition to the amino acid sequences and corresponding cTuberins described herein, the present disclosure further provides nucleic acid molecules encoding any one of the cTuberin proteins disclosed herein. In some embodiments, the nucleic acid molecule is codon optimized for expression in a human target cell. In some embodiments, the human target cell is a brain cell, heart cell, kidney cell, skin cell, or lung cell.

In some embodiments, the nucleic acid molecule is operably linked to a regulatory control sequence. In some embodiments, the regulatory control sequence comprises a human cytomegalovirus (CMV) promoter, a chicken β-actin (CBA) promoter, a Rous sarcoma virus (RSV) LTR promoter/enhancer, an SV40 promoter, a dihydrofolate reductase promoter, a phosphoglycerol kinase promoter, a CMV immediate/early gene enhancer/CBA promoter, a synapsin promoter, CMV-IE promoter/enhancer, a glial fibrillary acidic protein (GFAP) promoter, or a combination thereof. In some embodiments, the regulatory control sequence comprises a CMV immediate/early gene enhancer/CBA promoter and a woodchuck hepatitis virus posttranscriptional regulatory element (WPRE). In some embodiments, the regulatory control sequence comprises a beta-glucuronidase (GUSB) promoter. Further details regarding the GUSB promoter are provided in Shipley et al., Analysis of the 5′ Flanking Region of the Human β-Glucuronidase Gene, Genomics 10, 1009-1018 (1991), the contents of which are herein incorporated by reference in its entirety.

In some embodiments, the nucleic acid molecule comprises, or consists of, at least about 75% (for example, at least about 80%, about 85%, about 90%, about 95%, about 96%, about 97%, about 98%, about 99%, or greater, such as 100%) sequence identity to any one of SEQ ID NOs: 20-26. In some embodiments, the nucleic acid molecule comprises, or consists of, the sequence of any one of SEQ ID NOs: 20-26. In some embodiments, the nucleic acid molecule comprises, or consists of, the sequence of SEQ ID NO: 20. In some embodiments, the nucleic acid molecule comprises, or consists of, the sequence of SEQ ID NO: 21. In some embodiments, the nucleic acid molecule comprises, or consists of, the sequence of SEQ ID NO: 22. In some embodiments, the nucleic acid molecule comprises, or consists of, the sequence of SEQ ID NO: 23. In some embodiments, the nucleic acid molecule comprises, or consists of, the sequence of SEQ ID NO: 24. In some embodiments, the nucleic acid molecule comprises, or consists of, the sequence of SEQ ID NO: 25. In some embodiments, the nucleic acid molecule comprises, or consists of, the sequence of SEQ ID NO: 26.

The present disclosure provides nucleic acid molecules encoding a cTuberin comprising or consisting of (i) an N-terminal region capable of binding hamartin, and (ii) a C-terminal GTPase-activating protein (GAP) region, wherein the cTuberin lacks at least amino acid residues 451-932 of human tuberin (SEQ ID NO: 1); and wherein the nucleic acid molecule is operably linked to a regulatory control sequence comprising or consisting of a beta-glucuronidase (GUSB) promoter.

In some embodiments, the nucleic acid molecule encodes a cTuberin that lacks amino acid residues 451-1109 of human tuberin (SEQ ID NO: 1), and the nucleic acid molecule is operably linked to a regulatory control sequence comprising or consisting of a beta-glucuronidase (GUSB) promoter. In some embodiments, the nucleic acid molecule encodes a cTuberin that lacks amino acid residues 451-1139 of human tuberin (SEQ ID NO: 1), and the nucleic acid molecule is operably linked to a regulatory control sequence comprising or consisting of a beta-glucuronidase (GUSB) promoter. In some embodiments, the nucleic acid molecule encodes a cTuberin that lacks amino acid residues 451-1514 of human tuberin (SEQ ID NO: 1), and the nucleic acid molecule is operably linked to a regulatory control sequence comprising or consisting of a beta-glucuronidase (GUSB) promoter. In some embodiments, the nucleic acid molecule encodes a cTuberin that lacks amino acid residues 419-932 of human tuberin (SEQ ID NO: 1), and the nucleic acid molecule is operably linked to a regulatory control sequence comprising or consisting of a beta-glucuronidase (GUSB) promoter. In some embodiments, the nucleic acid molecule encodes a cTuberin that lacks amino acid residues 419-1109 of human tuberin (SEQ ID NO: 1), and the nucleic acid molecule is operably linked to a regulatory control sequence comprising or consisting of a beta-glucuronidase (GUSB) promoter. In some embodiments, the nucleic acid molecule encodes a cTuberin that lacks amino acid residues 419-1139 of human tuberin (SEQ ID NO: 1), and the nucleic acid molecule is operably linked to a regulatory control sequence comprising or consisting of a beta-glucuronidase (GUSB) promoter. In some embodiments, the nucleic acid molecule encodes a cTuberin that lacks amino acid residues 419-1514 of human tuberin (SEQ ID NO: 1), and the nucleic acid molecule is operably linked to a regulatory control sequence comprising or consisting of a beta-glucuronidase (GUSB) promoter. In some embodiments, the nucleic acid molecule encodes a cTuberin that lacks the amino acid residues of exon 25 of human tuberin (SEQ ID NO: 1). In some embodiments, the nucleic acid molecule encodes a cTuberin that lacks the amino acid residues of exon 30 of human tuberin (SEQ ID NO: 1). In some embodiments, the nucleic acid molecule encodes a cTuberin that lacks the amino acid residues of exon 33 of human tuberin (SEQ ID NO: 1). In some embodiments, the nucleic acid molecule encodes a cTuberin that lacks the amino acid residues of exons 25 and 30 of human tuberin (SEQ ID NO: 1). In some embodiments, the nucleic acid molecule encodes a cTuberin that lacks the amino acid residues of exons 25, 30, and 33 of human tuberin (SEQ ID NO: 1).

In some embodiments, the cTuberin lacks amino acid residues 419-932, 451-932, 419-1109, 419-1139, 451-1109, 451-1139, 419-1514, 451-1514, 419-1515, or 451-1515 of human tuberin (SEQ ID NO: 1): the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 6; and the nucleic acid molecule is operably linked to a regulatory control sequence comprising or consisting of a beta-glucuronidase (GUSB) promoter. In some embodiments, the cTuberin lacks amino acid residues 451-1514 or 451-1515 of human tuberin (SEQ ID NO: 1): the C-terminal region comprises, or consists of, an amino acid sequence with at least about 90% identity to SEQ ID NO: 6; and the nucleic acid molecule is operably linked to a regulatory control sequence comprising or consisting of a beta-glucuronidase (GUSB) promoter. In some embodiments, the cTuberin lacks amino acid residues 451-1514 or 451-1515 of human tuberin (SEQ ID NO: 1): the C-terminal region comprises, or consists of, the amino acid sequence of SEQ ID NO: 6; and the nucleic acid molecule is operably linked to a regulatory control sequence comprising or consisting of a beta-glucuronidase (GUSB) promoter.

In some embodiments, the cTuberin lacks amino acid residues 419-932, 451-932, 419-1109, 419-1139, 451-1109, or 451-1139 of human tuberin (SEQ ID NO: 1); the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 7; and the nucleic acid molecule is operably linked to a regulatory control sequence comprising or consisting of a beta-glucuronidase (GUSB) promoter. In some embodiments, the cTuberin lacks amino acid residues 419-932, 451-932, 419-1109, 419-1139, 451-1109, or 451-1139 of human tuberin (SEQ ID NO: 1): the C-terminal region comprises, or consists of, an amino acid sequence with at least about 90% identity to SEQ ID NO: 7; and the nucleic acid molecule is operably linked to a regulatory control sequence comprising or consisting of a beta-glucuronidase (GUSB) promoter. In some embodiments, the cTuberin lacks amino acid residues 419-932, 451-932, 419-1109, 419-1139, 451-1109, or 451-1139 of human tuberin (SEQ ID NO: 1); the C-terminal region comprises, or consists of, the amino acid sequence of SEQ ID NO: 7; and the nucleic acid molecule is operably linked to a regulatory control sequence comprising or consisting of a beta-glucuronidase (GUSB) promoter.

In some embodiments, the cTuberin lacks amino acid residues 419-932, 451-932, 419-1109, or 451-1109 of human tuberin (SEQ ID NO: 1): the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 8; and the nucleic acid molecule is operably linked to a regulatory control sequence comprising or consisting of a beta-glucuronidase (GUSB) promoter. In some embodiments, the cTuberin lacks amino acid residues 419-932, 451-932, 419-1109, or 451-1109 of human tuberin (SEQ ID NO: 1): the C-terminal region comprises, or consists of, an amino acid sequence with at least about 90% identity to SEQ ID NO: 8; and the nucleic acid molecule is operably linked to a regulatory control sequence comprising or consisting of a beta-glucuronidase (GUSB) promoter. In some embodiments, the cTuberin lacks amino acid residues 419-932, 451-932, 419-1109, or 451-1109 of human tuberin (SEQ ID NO: 1): the C-terminal region comprises, or consists of, the amino acid sequence of SEQ ID NO: 8; and the nucleic acid molecule is operably linked to a regulatory control sequence comprising or consisting of a beta-glucuronidase (GUSB) promoter.

In some embodiments, the cTuberin lacks amino acid residues 419-932 or 451-932 of human tuberin (SEQ ID NO: 1): the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 9; and the nucleic acid molecule is operably linked to a regulatory control sequence comprising or consisting of a beta-glucuronidase (GUSB) promoter. In some embodiments, the cTuberin lacks amino acid residues 419-932 or 451-932 of human tuberin (SEQ ID NO: 1): the C-terminal region comprises, or consists of, an amino acid sequence with at least about 90% identity to SEQ ID NO: 9; and the nucleic acid molecule is operably linked to a regulatory control sequence comprising or consisting of a beta-glucuronidase (GUSB) promoter. In some embodiments, the cTuberin lacks amino acid residues 419-932 or 451-932 of human tuberin (SEQ ID NO: 1): the C-terminal region comprises, or consists of, the amino acid sequence of SEQ ID NO: 9; and the nucleic acid molecule is operably linked to a regulatory control sequence comprising or consisting of a beta-glucuronidase (GUSB) promoter.

In some embodiments, the cTuberin lacks amino acid residues 419-932 or 451-932 of human tuberin (SEQ ID NO: 1): the C-terminal region comprises, or consists of, an amino acid sequence with at least about 75% (for example, at least about 80%, about 85%, about 90%, about 95%, about 96%, about 97%, about 98%, about 99%, or greater, such as 100%) identity to one of SEQ ID NOs: 10-12; and the nucleic acid molecule is operably linked to a regulatory control sequence comprising or consisting of a beta-glucuronidase (GUSB) promoter. In some embodiments, the cTuberin lacks amino acid residues 419-932 or 451-932 of human tuberin (SEQ ID NO: 1): the C-terminal region comprises, or consists of, an amino acid sequence with at least about 90% identity to one of SEQ ID NOs: 10-12; and the nucleic acid molecule is operably linked to a regulatory control sequence comprising or consisting of a beta-glucuronidase (GUSB) promoter. In some embodiments, the cTuberin lacks amino acid residues 419-932 or 451-932 of human tuberin (SEQ ID NO: 1); the C-terminal region comprises, or consists of, the amino acid sequence of one of SEQ ID NOs: 10-12; and the nucleic acid molecule is operably linked to a regulatory control sequence comprising or consisting of a beta-glucuronidase (GUSB) promoter.

In some embodiments, the cTuberin lacks amino acid residues 451-932, 451-1109, 451-1139, 451-1514, 451-1515 of human tuberin (SEQ ID NO: 1); the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 4; and the nucleic acid molecule is operably linked to a regulatory control sequence comprising or consisting of a beta-glucuronidase (GUSB) promoter. In some embodiments, the cTuberin lacks amino acid residues 451-932, 451-1109, 451-1139, 451-1514, 451-1515 of human tuberin (SEQ ID NO: 1); the N-terminal region comprises, or consists of, an amino acid sequence with at least about 90% identity to SEQ ID NO: 4; and the nucleic acid molecule is operably linked to a regulatory control sequence comprising or consisting of a beta-glucuronidase (GUSB) promoter. In some embodiments, the cTuberin lacks amino acid residues 451-932, 451-1109, 451-1139, 451-1514, or 451-1515 of human tuberin (SEQ ID NO: 1); the N-terminal region comprises, or consists of, the amino acid sequence of SEQ ID NO: 4; and the nucleic acid molecule is operably linked to a regulatory control sequence comprising or consisting of a beta-glucuronidase (GUSB) promoter.

In some embodiments, the cTuberin lacks amino acid residues 419-932, 419-1109, 419-1139, 419-1514, or 419-1515 of human tuberin (SEQ ID NO: 1); the N-terminal region comprises, or consists of, an amino acid sequence with at least about 75% (for example, at least about 80%, about 85%, about 90%, about 95%, about 96%, about 97%, about 98%, about 99%, or greater, such as 100%) identity to SEQ ID NO: 5; and the nucleic acid molecule is operably linked to a regulatory control sequence comprising or consisting of a beta-glucuronidase (GUSB) promoter. In some embodiments, the cTuberin lacks amino acid residues 419-932, 419-1109, 419-1139, 419-1514, or 419-1515 of human tuberin (SEQ ID NO: 1): the N-terminal region comprises, or consists of, an amino acid sequence with at least about 90% identity to SEQ ID NO: 5; and the nucleic acid molecule is operably linked to a regulatory control sequence comprising or consisting of a beta-glucuronidase (GUSB) promoter. In some embodiments, the cTuberin lacks amino acid residues 419-932, 419-1109, 419-1139, 419-1514, or 419-1515 of human tuberin (SEQ ID NO: 1): the N-terminal region comprises, or consists of, the amino acid sequence of SEQ ID NO: 5; and the nucleic acid molecule is operably linked to a regulatory control sequence comprising or consisting of a beta-glucuronidase (GUSB) promoter.

In some embodiments, the cTuberin lacks amino acid residues 451-1514 or 451-1515 of human tuberin (SEQ ID NO: 1): the C-terminal region comprises, or consists of, an amino acid sequence with at least 90% identity to SEQ ID NO: 6; the N-terminal region comprises, or consists of, an amino acid sequence with at least 90% identity to SEQ ID NO: 4; and the nucleic acid molecule is operably linked to a regulatory control sequence comprising or consisting of a beta-glucuronidase (GUSB) promoter. In some embodiments, the cTuberin lacks amino acid residues or 451-1514 or 451-1515 of human tuberin (SEQ ID NO: 1); the C-terminal region comprises, or consists of, the amino acid sequence of SEQ ID NO: 6; the N-terminal region comprises, or consists of, the amino acid sequence of SEQ ID NO: 4; and the nucleic acid molecule is operably linked to a regulatory control sequence comprising or consisting of a beta-glucuronidase (GUSB) promoter.

In some embodiments, the nucleic acid molecule comprises a nucleic acid sequence having at least about 75% (for example, at least about 80%, about 85%, about 90%, about 95%, about 96%, about 97%, about 98%, about 99%, or greater, such as 100%) sequence identity to any one of SEQ ID NOs: 20-26. In some embodiments, the nucleic acid molecule comprises a nucleic acid sequence having at least about 90% sequence identity to any one of SEQ ID NOs: 20-26.

In some embodiments, the nucleic acid molecule comprises an adeno-associated virus (AAV) expression cassette, the AAV expression cassette comprising from 5′ to 3′: a 5′ AAV inverted terminal repeat (ITR); any one of the nucleic acid molecules disclosed herein; and a 3′ AAV ITR. In some embodiments, the 5′ ITR and/or the 3′ ITR are derived from AAV2.

In some embodiments, the AAV expression cassettes disclosed herein comprise the cis-acting 5′ and 3′ inverted terminal repeat sequences, as described further in B. J. Carter, in “Handbook of Parvoviruses”, ed., P. Tijsser, CRC Press, pp. 155-168 (1990), which is incorporated herein by reference in its entirety for all purposes. The AAV ITR sequences may be obtained from any known AAV, including presently identified mammalian AAV types disclosed herein.

In some embodiments, the AAV expression cassette comprises a 5′ ITR and/or a 3′ ITR from AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV218, AAV2g9, AAV-LK03, AAV7m8, AAV Anc80, or AAV PHP.B. In some embodiments, the AAV expression cassette comprises a 5′ ITR from AAV2, a 3′ ITR from AAV2, or a combination thereof. In some embodiments, the AAV expression cassette comprises a 5′ ITR derived from AAV2, a 3′ ITR derived from AAV2, or a combination thereof.

In some embodiments, the 5′ AAV ITR sequence comprises, or consists of, a nucleic acid sequence having at least 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 27. In some embodiments, the 5′ AAV ITR sequence comprises, or consists of, the sequence of SEQ ID NO: 27.

In some embodiments, the 3′ AAV ITR sequence comprises, or consists of, a nucleic acid sequence having at least 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 28. In some embodiments, the 3′ AAV ITR sequence comprises, or consists of, the nucleic acid sequence of SEQ ID NO: 28.

In some embodiments, the AAV expression cassettes disclosed herein comprise additional expression control elements which are operably linked to the transgene. Expression control elements include, for example, appropriate transcription initiation, termination, and enhancer sequences: efficient RNA processing signals such as splicing and polyadenylation (polyA) signals: sequences that stabilize cytoplasmic mRNA: sequences that enhance translation efficiency: sequences that enhance protein stability; and sequences that enhance secretion of the encoded product.

In some embodiments, the AAV expression cassettes disclosed herein comprise an intron. In some embodiments, the intron is located between the promoter/enhancer sequence and the transgene. In some embodiments, the intron is derived from SV-40, and is referred to as the SV-40 T intron sequence. In some embodiments, the AAV expression cassettes disclosed herein comprise an internal ribosome entry site (IRES). In some embodiments, the AAV expression cassettes disclosed herein comprise a nucleic acid encoding a 2A self-cleaving peptide. Illustrative 2A self-cleaving peptides include P2A, E2A, F2A, and T2A. In some embodiments, the AAV expression cassettes disclosed herein comprise an element described in Sambrook et al, and references cited therein at, for example, pages 3.18 3.26 and 16.17 16.27 and Ausubel et al., Current Protocols in Molecular Biology, John Wiley & Sons, New York, 1989, each of which is incorporated herein by reference in its entirety for all purposes.

In some embodiments, the AAV expression cassettes disclosed herein comprise a woodchuck hepatitis virus post-transcriptional element (WPRE). (See, e.g., Wang and Verma, Proc. Natl. Acad. Sci., USA, 96: 3906-3910 (1999)). In some embodiments, the AAV expression cassettes disclosed herein comprise a hepatitis B virus posttranscriptional regulatory element (HBVPRE) or a RNA transport element (RTE). In some embodiments, the WPRE or HBVPRE sequence is any of the WPRE or HBVPRE sequences disclosed in U.S. Pat. No. 6,136,597 or 6,287,814, both of which are herein incorporated by reference in their entireties.

In some embodiments, the AAV expression cassettes disclosed herein comprise 5′ non-transcribed and 5′ non-translated sequences involved with the initiation of transcription and translation respectively, such as a TATA box, capping sequence, CAAT sequence, enhancer elements, and the like. In some embodiments, the AAV expression cassettes disclosed herein comprise an enhancer sequence or upstream activator sequence. In some embodiments, the AAV expression cassettes disclosed herein comprise 5′ leader or signal sequences.

In some embodiments, the AAV expression cassettes disclosed herein comprise a constitutive promoter. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1a promoter.

In some embodiments, the AAV expression cassettes disclosed herein comprise an inducible promoter. Non-limiting examples of inducible promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system and the rapamycin-inducible system. Other types of inducible promoters include those that are regulated by a specific physiological state, e.g., temperature, acute phase, a particular differentiation state of the cell, or a specific cell cycle phase.

In some embodiments, the AAV expression cassettes disclosed herein comprise the native promoter, or fragment thereof, or the native expression control element, operably linked to the transgene encoding cTuberin. In some embodiments, the AAV expression cassettes disclosed herein comprise regulatory sequences that impart tissue-specific gene expression capabilities. In some cases, the tissue-specific regulatory sequences bind tissue-specific transcription factors that induce transcription in a tissue specific manner. Examples of tissue-specific regulatory sequences include, but are not limited to the following tissue specific promoters: neuronal promoters such as the neuron-specific enolase (NSE) promoter, the neurofilament light chain gene promoter, and the neuron-specific vgf gene promoter.

In some embodiments, the AAV expression cassette comprises one or more promoters. In some embodiments, the AAV expression cassette comprises a chicken β-actin promoter. In some embodiments, the AAV expression cassette comprises a CB6 promoter. In some embodiments, the CB6 promoter comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 34. In some embodiments, the CB6 promoter comprises, or consists of, the nucleic acid sequence of SEQ ID NO: 34.

In some embodiments, the AAV expression cassette comprises a CMV-IE enhancer. In some embodiments, the enhancer is a CMV-IE enhancer. In some embodiments, the CMV-IE enhancer comprises a nucleic acid sequence having at least about 80% (for example, at least about 85%, about 90%, about 95%, about 96%, about 97%, about 98%, about 99%, about 99.5%, or greater, such as about 100%, including all values and subranges that lie therebetween) identity to the sequence of SEQ ID NO: 33. In some embodiments, the CMV-IE enhancer comprises, or consists of, the nucleic acid sequence of SEQ ID NO: 33.

In some embodiments, the AAV expression cassette comprises a consensus sequence, such as a Kozak sequence (for example, a DNA sequence transcribed to an RNA Kozak sequence). In some embodiments, the AAV expression cassette comprises a Kozak sequence. In some embodiments, the Kozak sequence comprises a nucleic acid sequence having at least about 80% (for example, at least about 85%, about 90%, about 95%, about 96%, about 97%, about 98%, about 99%, about 99.5%, or greater, such as 100%, including all values and subranges that lie therebetween) identity to the sequence of SEQ ID NO: 35. In some embodiments, the Kozak sequence comprises, or consists of, the nucleic acid sequence of SEQ ID NO: 35.

In some embodiments, the AAV expression cassettes disclosed herein comprise one or more binding sites for one or more microRNAs (miRNAs). In some embodiments, the AAV expression cassette comprises an miRNA binding site that is capable of regulating tissue specific expression of the cTuberin transgene. In some embodiments, the miRNA binding site that is capable of regulating tissue specific expression of the cTuberin transgene is an miR-122 binding site, an miR-133a, or a miR-1 binding site. For example, expression of the cTuberin transgene in the liver may be inhibited by incorporating a binding site for miR-122 such that mRNA expressed from the transgene binds to and is inhibited by miR-122 in the liver. Expression of the cTuberin transgene in the heart may be inhibited by incorporating a binding site for miR-133a or miR-1, such that mRNA expressed from the transgene binds to and is inhibited by miR-133a or miR-1 in the heart. In some embodiments, miRNA target sites in mRNA are in the 5′ untranslated region (UTR), the 3′ UTR, or in the coding region. Furthermore, the cTuberin transgene may be designed such that multiple miRNAs regulate mRNA by recognizing the same or multiple sites. The presence of multiple miRNA binding sites may result in the cooperative action of multiple RNA-induced silencing complexes (RISCs) and provide highly efficient inhibition of expression. The target site sequence may comprise a total of at least 5, 10, or more nucleotides, such as between 5-100, or between 10-60 nucleotides. The target site sequence may comprise at least 5 nucleotides of the sequence of a target gene binding site. In some embodiments, the AAV expression cassette comprises an miR-1 binding site, an miR-133a binding site, an miR-122 binding site, or any combination thereof.

In some embodiments, the AAV expression cassette comprises a polyadenylation (polyA) sequence. PolyA signals may be derived from many suitable species, including, without limitation SV-40, human and bovine. In some embodiments, the poly A sequence is a β-globin polyA sequence, such as a mammalian β-globin polyA sequence. In some embodiments, the polyA sequence is a human polyA sequence or a bovine β-globin poly A sequence. In some embodiments, the AAV expression cassette comprises a rabbit β-globin polyA sequence. In some embodiments, the rabbit β-globin polyA sequence comprises, or consists of, the nucleic acid sequence of SEQ ID NO: 36.

In some embodiments, the AAV expression cassette comprises from 5′ to 3″: (i) a 5′ AAV2-based ITR, (ii) a CMV-IE enhancer, (iii) a CB6 promoter, (iv) a transgene encoding any one of the cTuberin proteins disclosed herein, (v) a polyadenylation sequence, and (vi) a 3′ AAV2-based ITR. In some embodiments, the AAV expression cassette comprises from 5′ to 3′: (i) a 5′ AAV2-based ITR comprising a nucleic acid sequence of SEQ ID NO: 27, (ii) a CMV-IE enhancer comprising a nucleic acid sequence of SEQ ID NO: 33, (iii) a CB6 promoter comprising a nucleic acid sequence of SEQ ID NO: 34: (iv) a transgene encoding any one of the cTuberin proteins disclosed herein, (v) a polyadenylation sequence, and (vi) a 3′ AAV2-based ITR comprising a nucleic acid sequence of SEQ ID NO: 28.

In some embodiments, the AAV expression cassette comprises from 5′ to 3″: (i) a 5′ AAV2-based ITR, (ii) a CB6 promoter, (iii) a transgene encoding any one of the cTuberin proteins disclosed herein, (iv) a polyadenylation sequence, and (v) a 3′ AAV2-based ITR. In some embodiments, the AAV expression cassette comprises from 5′ to 3″: (i) a 5′ AAV2-based ITR comprising a nucleic acid sequence of SEQ ID NO: 27, (ii) a CB6 promoter comprising a nucleic acid sequence of SEQ ID NO: 34, (iii) a transgene encoding any one of the cTuberin proteins disclosed herein, (iv) a polyadenylation sequence, and (v) a 3′ AAV2-based ITR comprising a nucleic acid sequence of SEQ ID NO: 28.

In some embodiments, the AAV expression cassette comprises from 5′ to 3″: (i) a 5′ AAV2-based ITR, (ii) a GUSB promoter, (iii) a transgene encoding any one of the cTuberin proteins disclosed herein, (iv) a polyadenylation sequence, and (v) a 3′ AAV2-based ITR. In some embodiments, the AAV expression cassette comprises from 5′ to 3″: (i) a 5′ AAV2-based ITR comprising a nucleic acid sequence of SEQ ID NO: 27, (ii) a GUSB promoter, (iii) a transgene encoding any one of the cTuberin proteins disclosed herein, (iv) a polyadenylation sequence and (v) a 3′ AAV2-based ITR comprising a nucleic acid sequence of SEQ ID NO: 28.

Recombinant Adeno-Associated Virus (rAAV) for Treating Tuberous Sclerosis

The present disclosure also provides plasmids, comprising any one of the nucleic acid molecules disclosed herein, and host cells comprising any one of the nucleic acid molecules or plasmids disclosed herein.

The present disclosure further provides methods of producing a recombinant adeno-associated virus (rAAV). In some embodiments, a method of producing an rAAV comprises contacting a host cell with any one of the nucleic acid molecules or plasmids disclosed herein. Accordingly, the present disclosure further provides recombinant adeno-associated viruses (rAAVs) produced by the methods of producing rAAVs disclosed herein.

The present disclosure also provides rAAVs. In some embodiments, an rAAV comprises an AAV capsid protein, and any one of the nucleic acid molecules or AAV expression cassettes disclosed herein. The present disclosure also provides compositions, comprising any one of the cTuberin proteins, any one of the nucleic acid molecules, any one of the plasmids, any one of the host cells, or any one of the rAAVs disclosed herein.

In some embodiments, the rAAV comprises an AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu. 68, avian AAV, bovine AAV, canine AAV, equine AAV, ovine AAV, snake AAV, bearded dragon AAV, AAV2i8, AAV2g9, AAV-LK03, AAV7m8, AAV Anc80, or AAV PHP.B capsid protein.

In some embodiments, the rAAV comprises an AAV9 capsid protein, an AAV8 capsid protein and/or an AAVrh10 capsid protein. In some embodiments, the rAAV comprises an AAV9) capsid protein. In some embodiments, the rAAV comprises an AAV8 capsid protein.

In some embodiments, the rAAV comprises an AAVrh10 capsid protein. In some embodiments, the rAAV is a pseudotyped AAV, comprising the AAV capsid protein of one serotype and the AAV ITRs derived from a different serotype. In some embodiments, the rAAV comprises a chimeric AAV capsid, or a humanized AAV capsid. In some embodiments, the rAAV is a self-complementary AAV (scAAV). In some embodiments, the rAAV is a single-stranded AAV.

In some embodiments, preparation of rAAV particles involves culturing a host cell that contains a nucleic acid sequence encoding an AAV capsid protein or fragment thereof: a functional rep gene: a recombinant AAV vector composed of AAV inverted terminal repeats (ITRs) and the AAV expression cassette encoding any one of the cTuberin proteins disclosed herein; and sufficient helper functions to permit packaging of the recombinant AAV vector into the AAV capsid proteins. In some embodiments, the components to be cultured in the host cell to package an rAAV vector in an AAV capsid are provided to the host cell in trans. In some embodiments, any one or more of the required components (e.g., recombinant AAV vector, rep sequences, cap sequences, and/or helper functions) are provided by a stable host cell that has been engineered to contain one or more of the required components.

In some embodiments, a stable host cell will contain the required component(s) under the control of an inducible promoter or a constitutive promoter. In some embodiments, a selected stable host cell contains selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. The recombinant AAV vector, rep sequences, cap sequences, and helper functions required for producing the rAAVs disclosed herein may be delivered to the packaging host cell using any appropriate genetic element (for example, a vector). Further details on methods of preparing rAAV particles are provided in Sambrook et al., Molecular Cloning: A Laboratory Manual, Cold Spring Harbor Press, Cold Spring Harbor, N.Y.: K. Fisher et al, J. Virol., 70:520-532 (1993) and U.S. Pat. No. 5,478,745, the contents of each of which are herein incorporated in its entirety for all purposes.

In some embodiments, recombinant AAVs are produced using the triple transfection method, as described in U.S. Pat. No. 6,001,650, the contents of which are herein incorporated in its entirety for all purposes. In some embodiments, the recombinant AAVs are produced by transfecting a host cell with a recombinant AAV vector (comprising the AAV expression cassette encoding cTuberin) to be packaged into AAV particles, an AAV helper function vector, and an accessory function vector. An AAV helper function vector encodes the “AAV helper function” sequences (i.e., rep and cap), which function in trans for productive AAV replication and encapsidation. Non-limiting examples of AAV helper function vectors include pHLP19 and pRep6cap6 vector, described in U.S. Pat. Nos. 6,001,650 and 6,156,303, respectively, the contents of each of which are herein incorporated in its entirety for all purposes. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some embodiments, recombinant AAVs are produced using baculovirus vectors. Baculovirus vectors are used to produce recombinant AAVs in insect cells (e.g., Spodoptera frugiperda (Sf9) cells). Further details regarding the production of AAVs encoding cTuberin is found in U.S. Patent Publication No. 2020/0079824, the contents of which are herein incorporated by reference in its entirety for all purposes.

### Pharmaceutical Compositions

The present disclosure further provides pharmaceutical compositions, comprising: (a) any one of the nucleic acid molecules disclosed herein, any one of the plasmids disclosed herein, any one of the host cells disclosed herein, or any one the rAAVs disclosed herein; and (b) a pharmaceutically acceptable carrier.

In some embodiments, the compositions disclosed herein comprise at least one pharmaceutically acceptable carrier, excipient, and/or vehicle, for example, solvents, buffers, solutions, dispersion media, coatings, antibacterial agents, antifungal agents, isotonic agents, and absorption delaying agents. In some embodiments, the pharmaceutically acceptable carrier, excipient, and/or vehicle comprises saline, buffered saline, dextrose, water, glycerol, sterile isotonic aqueous buffer, or a combination thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like), or a suitable mixture thereof. In some embodiments, the compositions disclosed herein further comprise emulsifying or wetting agents, or pH buffering agents. Such species may be present in small amounts (e.g., less than 10% by weight of the composition, such as less than 5% by weight of the composition, 2% by weight of the composition, 1% by weight of the composition, or less).

In some embodiments, the compositions disclosed herein further comprise one or more other pharmaceutical ingredients, such as one or more preservatives or chemical stabilizers. Examples of preservatives and chemical stabilizers include, but are not limited to, chlorobutanol, potassium sorbate, sorbic acid, sulfur dioxide, propyl gallate, the parabens, ethyl vanillin, glycerin, phenol, parachlorophenol, and albumin. In some embodiments, the compositions disclosed herein may further comprise antibacterial agents and/or antifungal agents, such as, parabens, chlorobutanol, phenol, sorbic acid, and thimerosal: isotonic agents, such as sugars and sodium chloride and/or agents delaying absorption, such as aluminum monostearate and gelatin. In some embodiments, the compositions disclosed herein comprise a surfactant, such as, pluronic F68 (Poloxamer 188, also known as LUTROL® F68).

In some embodiments, the compositions disclosed herein are formulated to reduce aggregation of AAV particles in the composition, particularly where high rAAV concentrations are present (e.g., ˜10¹³ GC/ml or more). Methods for reducing aggregation of rAAVs include addition of surfactants, pH adjustment, and salt concentration adjustment, as further described in Wright, et al., Molecular Therapy (2005) 12, 171-178, the contents of which are incorporated herein by reference in its entirety for all purposes.

In some embodiments, the pharmaceutical compositions are in a form of an injectable solution or dispersion, such as an aqueous solution or dispersion. In some embodiments, the pharmaceutical composition is a sterile powder for the extemporaneous preparation of sterile injectable solutions or dispersions. Dispersions may be prepared in water, glycerol, liquid polyethylene glycols, oils, or any combination thereof. Delivery vehicles such as liposomes, nanocapsules, microparticles, microspheres, lipid particles, vesicles, and the like, may be used for the introduction of the pharmaceutical compositions disclosed herein.

The present disclosure also provides extracellular vesicles (EVs) comprising any one of the cTuberin proteins disclosed herein, or any nucleic acid molecules disclosed herein. In some embodiments, the pharmaceutical compositions comprise extracellular vesicles (EVs) comprising any one of the cTuberin proteins disclosed herein, or any nucleic acid molecule disclosed herein. Extracellular vesicles, including but not limited to exosomes, microvesicles, microparticles, circulating microvesicles, shedding microvesicles, nanovesicles, nanoparticles, apoptotic bodies, and membrane vesicles, are fragments of plasma membrane ranging from, for example, 20 nanometers (nm) to 10 micrometers (μm), shed from almost all cell types. In some embodiments, EVs are isolated and purified using filtration, differential centrifugation, ultracentrifugation, flotation of vesicles in gradients (sucrose, OptiPrep™), and immunoaffinity capture utilizing antibodies against membrane proteins. Further details are provided in Simpson R J, Mathivanan S (2012) Extracellular Microvesicles: The Need for Internationally Recognised Nomenclature and Stringent Purification Criteria. J Proteomics: van der Pol et al., Classification, functions, and clinical relevance of extracellular vesicles, Pharmacol Rev. 2012 July; 64(3):676-705: Raposo and Stoorvogel, Extracellular vesicles: exosomes, microvesicles, and friends, J Cell Biol. 2013 Feb. 18; 200(4):373-83; and Witwer et al., Standardization of sample collection, isolation and analysis methods in extracellular vesicle research, J Extracell Vesicles. 2013 May 27; 2, the contents of each of which are incorporated herein by reference in their entireties for all purposes.

### Methods of Treating Tuberous Sclerosis

The present disclosure also provides methods of expressing any one of the cTuberins disclosed herein in a target cell, comprising: contacting any one of the nucleic acid molecules disclosed herein, any one of the plasmids disclosed herein, any one of the rAAVs disclosed herein, any one of the EVs disclosed herein, or any one of the compositions disclosed herein with the target cell, thereby expressing cTuberin in the target cell.

In addition, the present disclosure provides methods of inhibiting mTORC1 and/or Rheb activity, comprising: contacting any one of the nucleic acid molecules disclosed herein, any one of the plasmids disclosed herein, any one of the rAAVs disclosed herein, any one of the EVs disclosed herein, or any one of the compositions disclosed herein with the target cell, thereby inhibiting mTORC1 and/or Rheb activity in the target cell.

In some embodiments, the target cell is a brain cell, heart cell, kidney cell, skin cell, lung cell, or any combination thereof. In some embodiments, the contacting step is performed in vitro, ex vivo, or in vivo. In some embodiments, the contacting step is performed in vivo in a subject in need thereof. In some embodiments, the contacting step comprises administering a therapeutically effective amount of the nucleic acid molecule, the plasmid, the rAAV, or the composition to the subject.

The present disclosure also provides methods of treating tuberous sclerosis or a symptom thereof in a subject in need thereof, comprising: administering to the subject a therapeutically effective amount of any one of the nucleic acid molecules disclosed herein, any one of the plasmids disclosed herein, any one of the rAAVs disclosed herein, any one of the EVs disclosed herein, or any one of the compositions disclosed herein, thereby treating tuberous sclerosis in the subject. In a related aspect, the present disclosure provides methods of ameliorating, diminishing the severity of, eliminating, and/or delaying the onset of one or more symptoms of tuberous sclerosis in a subject in need thereof, comprising: administering to the subject a therapeutically effective amount of any one of the nucleic acid molecules disclosed herein, any one of the plasmids disclosed herein, any one of the rAAVs disclosed herein, or any one of the compositions disclosed herein, thereby ameliorating, diminishing the severity of, eliminating, and/or delaying the onset of the one or more symptoms of tuberous sclerosis in the subject.

The present disclosure further provides methods of treating a renal angiomyolipoma or a symptom thereof in a subject in need thereof, comprising: administering to the subject a therapeutically effective amount of any one of the nucleic acid molecules disclosed herein, any one of the plasmids disclosed herein, any one of the rAAVs disclosed herein, any one of the EVs disclosed herein, or any one of the compositions disclosed herein, thereby treating the renal angiomyolipoma in the subject. In some embodiments, treatment of a renal angiomyolipoma results in regression, shrinkage, elimination, or delayed growth of the renal angiomyolipoma.

The present disclosure additionally provides methods of treating lymphangioleiomyomatosis (LAM) or a symptom thereof in a subject in need thereof, comprising: administering to the subject a therapeutically effective amount of any one of the nucleic acid molecules disclosed herein, any one of the plasmids disclosed herein, any one of the rAAVs disclosed herein, any one of the EVs disclosed herein, or any one of the compositions disclosed herein, thereby treating the LAM in the subject. In a related aspect, the present disclosure provides methods of ameliorating, diminishing the severity of, eliminating, and/or delaying the onset of one or more symptoms of lymphangioleiomyomatosis (LAM) in a subject in need thereof, comprising: administering to the subject a therapeutically effective amount of any one of the nucleic acid molecules disclosed herein, any one of the plasmids disclosed herein, any one of the rAAVs disclosed herein, or any one of the compositions disclosed herein, thereby ameliorating, diminishing the severity of, eliminating, and/or delaying the onset of the one or more symptoms of LAM in the subject.

The present disclosure also provides methods of treating brain dysfunction in a subject in need thereof, comprising: administering to the subject a therapeutically effective amount of any one of the nucleic acid molecules disclosed herein, any one of the plasmids disclosed herein, any one of the rAAVs disclosed herein, any one of the EVs disclosed herein, or any one of the compositions disclosed herein, thereby treating the brain dysfunction in the subject. In a related aspect, the present disclosure provides methods of ameliorating, diminishing the severity of, eliminating, and/or delaying the onset of one or more symptoms of brain dysfunction in a subject in need thereof, comprising: administering to the subject a therapeutically effective amount of any one of the nucleic acid molecules disclosed herein, any one of the plasmids disclosed herein, any one of the rAAVs disclosed herein, or any one of the compositions disclosed herein, thereby ameliorating, diminishing the severity of, eliminating, and/or delaying the onset of the one or more symptoms of brain dysfunction in the subject.

In some embodiments, the tuberous sclerosis is associated with, correlated with, or caused by a decrease in the activity of tuberin. In some embodiments, the subject suffers from tuberous sclerosis. In some embodiments, the subject is at a risk of developing at least one symptom of tuberous sclerosis.

In some embodiments, the subject has been diagnosed with tuberous sclerosis. In some embodiments, the subject is diagnosed as having tuberous sclerosis based on clinical criteria, such as, the presence of seizures; delayed development; white patches on the skin (hypomelanotic macules); the identification of cardiac tumor rhabdomyoma; the identification of tumors in the brain, heart, liver, or kidneys; examination of the skin for a wide variety of skin features; the fingernails, and toenails for ungual fibromas; the teeth and gums for dental pits and/or gum fibromas; the eyes for retinal lesions; facial angiofibromas; and/or the presence of hypomelanotic macules. In some embodiments, the subject is a human subject. In some embodiments, the subject is less than 18 years of age, such as between 12 to 18 years of age, between 8 to 12 years of age, between 6 to 12 years of age, between 2 to 18 years of age, between 0) to 2 years of age, or any range therein. In some embodiments, the subject is a neonate or an infant. In some embodiments, an infant is diagnosed with tuberous sclerosis based on clinical criteria, such as, the presence of cardiac rhabdomyomas at birth or infantile spasms in the first six months of life.

In some embodiments, the tuberous sclerosis is associated with, correlated with, or caused by mutations in the TSC2 gene. In some embodiments, the subject has a mutation in the TSC2 gene. In some embodiments, the subject has a mutation in one or both alleles of TSC2 in at least one cell in the body. In some embodiments, the mutation is an inherited germline mutation. In some embodiments, the mutation is a somatic mutation. In some embodiments, the mutation in one allele of TSC2 is an inherited mutation and the mutation in the second allele of TSC2 is a somatic mutation. In some embodiments, the subject at risk for developing at least one symptom of tuberous sclerosis is a subject having an inherited germline mutation in one allele of TSC2. In some embodiments, the subject at risk for developing at least one symptom of tuberous sclerosis is a subject whose one or both parents is a carrier of one or more mutant tuberin gene alleles.

In some embodiments, the subject has a mutation in both alleles of TSC2 in at least one cell in the body. In some embodiments, the subject has a mutation in both alleles of TSC2 in at least one cell in the brain, heart, kidney, skin, lung, and/or other organs. In some embodiments, the subject is homozygous, or compound heterozygous for the mutation in TSC2.

In some embodiments, the subject has a mutation in the TSC1 gene. In some embodiments, the subject has a mutation in the TSC1 gene and a mutation in the TSC2 gene.

In some embodiments, the subject is diagnosed as having tuberous sclerosis or at risk for developing tuberous sclerosis by testing for the presence of any one or more of mutations in TSC2 described herein in a biological sample derived from the subject. In some embodiments, the mutation in the TSC2 gene may be any amino acid modification, such as, for example, amino acid insertions, deletions, splice site mutations, and/or amino acid substitutions. Details regarding the mutations in TSC2 gene are described further in Reyna-Fabián, M.E., Sci Rep 10, 6589 (2020), Gilbert J R, et al., Neurogenetics. 1998 August; 1(4):267-72; Avgeris, S., Sci Rep 7, 16697 (2017); and Rosset C, et al., Genet Mol Biol. 2017; 40(1):69-79, the contents of each of which is herein incorporated in their entireties for all purposes. In some embodiments, the subject has a mutation in exon 33, exon 37, and/or exon 38 of the TSC2 gene. In some embodiments, the subject has a mutation in exon 33 of the TSC2 gene.

In some embodiments, the administration of a therapeutically effective amount of a therapeutic provided herein (e.g., a therapeutically effective amount of any one of the nucleic acid molecules disclosed herein, any one of the plasmids disclosed herein, any one of the rAAVs disclosed herein, or any one of the compositions disclosed herein) diminishes the severity of any one of the symptoms of tuberous sclerosis disclosed herein. In some embodiments, the administration of a therapeutically effective amount of a therapeutic provided herein delays the onset of any one of the symptoms of tuberous sclerosis disclosed herein. In some embodiments, the administration of a therapeutically effective amount of a therapeutic provided herein eliminates a symptom of any one of the symptoms of tuberous sclerosis disclosed herein. In some embodiments, the administration of a therapeutically effective amount of a therapeutic provided herein ameliorates a symptom of any one of the symptoms of tuberous sclerosis disclosed herein. In some embodiments, the symptom of tuberous sclerosis is the presence of any one or more of the following: tumor or hamartoma in the brain, heart, liver, kidneys, eyes or skin; subependymal outgrowths or nodules; subependymal giant cell astrocytomas; cortical tubers; brain dysfunction; seizures; delayed development; hypomelanotic macules; cardiac tumor rhabdomyoma; ungual fibromas; dental pits and/or gum fibromas; retinal lesions; facial angiofibromas; renal angiomyolipomas; lymphangioleiomyomatosis (LAM); internal bleeding; autism; epilepsy; hydrocephalus; and/or the presence of hypomelanotic macules. In some embodiments, the symptom of tuberous sclerosis is any one described in Randle S C., Pediatr Ann. 2017 Apr. 1; 46(4):e166-e171, Uysal S P, Turk J Med Sci. 2020 Nov. 3; 50(SI-2):1665-1676, and Henske E P, et al. Nat Rev Dis Primers. 2016 May 26; 2:16035, the contents of each of which are herein incorporated by reference in their entireties for all purposes.

In some embodiments, the administration is associated with, correlated with, or results in a decrease in the size of a tumor or hamartoma in the subject. In some embodiments, the administration is associated with, correlated with, or results in an at least about 5% (for example, at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or greater, such as 100%) decrease in the size of a tumor or hamartoma in the subject, as compared to a control subject with tuberous sclerosis who is not administered the compositions disclosed herein.

In some embodiments, the administration is associated with, correlated with, or results in a decrease in the number of tumors or hamartomas in the subject. In some embodiments, the administration is associated with, correlated with, or results in an at least about 5% (for example, at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or greater, such as 100%) decrease in the number of tumors or hamartomas in the subject, as compared to a control subject with tuberous sclerosis who is not administered the compositions disclosed herein.

Without being bound by a theory, it is thought that while administration of rapamycin to subjects with tuberous sclerosis has been associated with adverse effects due to over-inhibition of mTORC1, the expression of cTuberin using the compositions and methods disclosed herein may not cause over-inhibition of mTORC1. This is because subjects with tuberous sclerosis associated with mutations in TSC2 may have normal levels of hamartin, which would ensure normal inhibition of mTORC1. Furthermore, while rapamycin and its analogues can inhibit just mTORC1, the expression of cTuberin using the compositions disclosed results in mTORC1-independent Rheb-dependent pathological effects, in addition to the inhibition of mTORC1. Therefore, the compositions and methods disclosed herein can be more efficacious than rapamycin-based therapies for tuberous sclerosis.

In some embodiments, the method comprises administering a therapeutically effective amount of rAAV, wherein the therapeutically effective amount is in a range of about 10⁵ genome copies to 10²⁰ genome copies per kilogram (kg), for example, about 10⁶ genome copies/kg, about 10⁷ genome copies/kg, about 10⁸ genome copies/kg, about 10⁹ genome copies/kg, about 10¹⁰ genome copies/kg, about 10¹¹ genome copies/kg, about 10¹² genome copies/kg, about 10¹³ genome copies/kg, about 10¹⁴ genome copies/kg, about 10¹⁵ genome copies/kg, about 10¹⁶ genome copies/kg, about 10¹⁷ genome copies/kg, about 10¹⁸ genome copies/kg, or about 10¹⁹ genome copies/kg, including all values and subranges that lie therebetween. In some embodiments, the method comprises administering a therapeutically effective amount of rAAV, wherein the therapeutically effective amount is in a range of 10¹⁰ genome copies to 10¹⁴ genome copies per kilogram. In some embodiments, the method comprises administering a therapeutically effective amount of rAAV, wherein the therapeutically effective amount is in a range of 10⁹ genome copies to 10¹⁵ genome copies per kilogram.

In some embodiments, the therapeutically effective amount is in the range of about 10⁵ to 10²⁰ genome copies per subject, for example, about 10⁶ genome copies per subject, about 10⁷ genome copies per subject, about 10⁸ genome copies per subject, about 10⁹ genome copies per subject, about 10¹⁰ genome copies per subject, about 10¹¹ genome copies per subject, about 10¹² genome copies per subject, about 10¹³ genome copies per subject, about 10¹⁴ genome copies per subject, about 10¹⁵ genome copies per subject, about 10¹⁶ genome copies per subject, about 10¹⁷ genome copies per subject, about 10¹⁸ genome copies per subject, or about 10¹⁹ genome copies per subject, including all values and subranges that lie therebetween. In some embodiments, the therapeutically effective amount is in the range of about 10⁹ to 10¹⁶ genome copies per subject.

In some embodiments, the therapeutically effective amount is administered in a volume of about 1 microliters (μl) to about 100 mL of solution, for example, about 10 μl, about 50 μl, about 100 μl, about 125 μl, about 150 μl, about 175 μl, about 200 μl, about 250 μl, about 300 μl, about 350 μl, about 400 μl, about 450 μl, about 500 μl, about 550 μl, about 600 μl, about 650 μl, about 700 μl, about 750 μl, about 800 μl, about 850 μl, about 900 μl, about 950 μl, about 1 milliliters (mL), about 20 mL, about 30 mL, about 40 mL, about 50 mL, about 60 mL, about 70 mL, about 80 mL, about 90 mL, or about 100 mL, including all values and subranges that lie therebetween. The volume used may depend on the dose of the rAAV, and the route of administration. For example, for intrathecal or intracerebral administration a volume in the range of about 1 μl to about 10 μl, or about 10 μl to about 100 μl may be used. For intravenous administration a volume in range of about 10 μl to about 100 μl, or about 100 μl to 1 mL, or about 1 mL to about 10 mL, or more may be used.

In some embodiments, more than one administration (e.g., two, three, four, or more administrations) may be employed to achieve the desired level of gene expression over a period of various intervals, e.g., daily, weekly, monthly, yearly, etc.

In some embodiments, the administration is by injection into the central nervous system. Other modes of administration that may be used include dermal, oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). In some embodiments, the administration is by intracerebroventricular, or intracranial injection. In some embodiments, the cTuberin, the nucleic acid molecule, the plasmid, the composition, or the rAAV is administered intravascularly, into the renal artery or vein, into the lungs, into the cisterna magna, intracerebrally, intrathecally, intravenously, intraventricularly, intracerebroventricularly, intraperitoneally, or dermally.

In some embodiments, the method comprises administering a therapeutically effective amount of the cTuberin, the nucleic acid molecule, the plasmid, the composition, or the rAAV to a subject with renal angiomyolipoma by intravascular injection, for example, into the renal artery or vein. In some embodiments, a therapeutically effective amount of the cTuberin, the nucleic acid molecule, the plasmid, the composition, or the rAAV is targeted to the renal angiomyolipoma. In some embodiments, the method comprises administering a therapeutically effective amount of the cTuberin, the nucleic acid molecule, the plasmid, the composition, or the rAAV to a subject with lymphangioleiomyomatosis (LAM) by intravascular injection. In some embodiments, a therapeutically effective amount of the cTuberin, the nucleic acid molecule, the plasmid, the composition, or the rAAV is targeted to the LAM. In some embodiments, the subject has a brain dysfunction. In some embodiments, the cTuberin, the nucleic acid molecule, the plasmid, the composition, or the rAAV is provided to the subarachnoid space.

In some embodiments, the cTuberin, the nucleic acid molecule, the plasmid, the composition, or the rAAV is provided (e.g., administered) to a brain cell, a heart cell, a kidney cell, a skin cell, or a lung cell. In some embodiments, the nucleic acid molecule, the plasmid, the host cell, the rAAV, or the composition is administered into the bloodstream of the subject. Administration into the bloodstream may be by injection into a vein, an artery, or any other vascular conduit. In some embodiments, the nucleic acid molecule, the plasmid, the cell, the rAAV, or the composition is administered intravascularly. In some embodiments, the nucleic acid molecule, the plasmid, the host cell, the rAAV, or the composition is administered intravenously.

In some embodiments, the nucleic acid molecule, the plasmid, the host cell, the rAAV, or the composition is delivered to brain tissue, meninges, neuronal cells, glial cells, astrocytes, oligodendrocytes, cerebrospinal fluid (CSF), interstitial spaces, or the like. In some embodiments, recombinant AAVs may be delivered directly to the spinal cord or brain by injection into the ventricular region, as well as to the striatum (e.g., the caudate nucleus or putamen of the striatum), and neuromuscular junction, or cerebellar lobule, with a needle, catheter or related device, using neurosurgical techniques, such as by stereotactic injection.

In some embodiments, the administration may comprise administering the nucleic acid molecule, the plasmid, the host cell, the rAAV, or the composition by more than route—concurrently or at different time points.

In some embodiments, the methods disclosed herein comprise administering another secondary therapy to subject. In some embodiments, secondary therapy comprises administration of an anti-seizure drug. Non-limiting examples of anti-seizure drugs include Carbamazepine, Phenytoin, Valproic acid, Oxcarbazepine, Lamotrigine, Gabapentin, Topiramate, and Phenobarbital, and Zonisamide. In some embodiments, secondary therapy comprises administration of rapamycin or its analogues. The secondary therapy may be administered sequentially, or concurrently to the subject.

The present disclosure also provides kits comprising one or more agents (e.g., any one of the nucleic acid molecules disclosed herein, any one of the plasmids disclosed herein, any one of the rAAVs disclosed herein, or any one of the compositions disclosed herein). In some embodiments, the kits are pharmaceutical or diagnostic or research kits to be used in therapeutic, diagnostic or research applications. A kit may include one or more containers housing the agents disclosed herein and instructions for use. In certain embodiments, agents in a kit are in a pharmaceutical formulation and dosage suitable for a particular application and for a method of administration of the agents. In some embodiments, the container is a syringe, vial, tube, topical application devices, IV needle tubing and bag, or another container.

In some embodiments, the kit contains a first pharmaceutical composition, comprising a cTuberin-encoding nucleic acid molecule (e.g., in an rAAV, as described herein), or a cTuberin, and a second pharmaceutical composition, comprising one or more drugs used for the treatment of tuberous sclerosis, such as, rapamycin and its analogues. In some embodiments, the kit includes instructions for administering the two compositions sequentially or concurrently, or mixing the two pharmaceutical compositions prior to administration.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

Unless the context indicates otherwise, it is specifically intended that the various features described herein can be used in any combination.

### EXAMPLES

### Example 1: Generation of New Condensed Tuberin (cTuberin) Constructs

Various cTuberin proteins comprising deletions of specific amino acid residues are generated by cloning different nucleic acid constructs into suitable expression plasmids. The constructs and the amino acid deletions are listed in Table 1 below.

TABLE 1

| | | | N-terminal region | | C-terminal region | | | | | |
|---|---|---|---|---|---|---|---|---|---|---|
| | | | Amino acid | | Amino acid | | No. of amino acids | Deleted | | Full |
| | Upstream | | residues | | residues | | from the C- | amino acids | | sequence of |
| | Response | | relative to | | relative to | | terminus of SEQ | relative to | Full | nucleic acid |
| | Element | | SEQ ID | | SEQ ID | | ID NO: 1 included | SEQ ID | sequence of | encoding |
| Plasmid | (URE) | Promoter | NO: 1 | Sequence | NO: 1 | Sequence | in the variant | NO: 1 | cTuberin | cTuberin |
| V2 | CMV-IE | CB6 | 1-450 | SEQ ID | 1140-1807 | SEQ ID | 668 | 451-1139 | SEQ ID | SEQ ID |
| | | | | NO: 4 | | NO: 7 | | | NO: 14 | NO: 21 |
| V3 | CMV-IE | CB6 | 1-418 | SEQ ID | 1110-1807 | SEQ ID | 698 | 419-1109 | SEQ ID | SEQ ID |
| | | | | NO: 5 | | NO: 8 | | | NO: 15 | NO: 22 |
| V4 | CMV-IE | CB6 | 1-418 | SEQ ID | 933-1807 | SEQ ID | 875 | 419-932 | SEQ ID | SEQ ID |
| | | | | NO: 5 | | NO: 9 | | | NO: 16 | NO: 23 |
| V5 | | CB6 | 1-418 | SEQ ID | 933-1807 | SEQ ID | 875 | 419-932 | SEQ ID | SEQ ID |
| | | | | NO: 5 | | NO: 9 | | | NO: 16 | NO: 23 |
| V6 | CMV-IE | CB6 | 1-418 | SEQ ID | 933-946 and | SEQ ID | 875 Δ25 | 419-932, | SEQ ID | SEQ ID |
| | | | | NO: 5 | 989-1807 | NO: 10 | | 947-988 | NO: 17 | NO: 24 |
| V7 | CMV-IE | CB6 | 1-418 | SEQ ID | 933-946, | SEQ ID | 875 Δ25 Δ30 | 419-932, | SEQ ID | SEQ ID |
| | | | | NO: 5 | 989-1204, | NO: 11 | | 947-988, | NO: 18 | NO: 25 |
| | | | | | 1272-1807 | | | 1205-1271 | | |
| V8 | CMV-IE | CB6 | 1-418 | SEQ ID | 933-946, | SEQ ID | 875 Δ25 Δ30 Δ33 | 419-932, | SEQ ID | SEQ ID |
| | | | | NO: 5 | 989-1204, | NO: 12 | | 947-988, | NO: 19 | NO: 26 |
| | | | | | 1272-1335, | | | 1205-1271, | | |
| | | | | | 1498-1807 | | | 1336-1497 | | |
| V9 | | GUSB | 1-450 | SEQ ID | 1516-1807 | SEQ ID | 292 | 451-1515 | SEQ ID | SEQ ID |
| | | | | NO: 4 | | NO. 6 | | | NO: 13 | NO: 20 |
| V10 | | GUSB | 1-450 | SEQ ID | 1140-1807 | SEQ ID | 668 | 451-1139 | SEQ ID | SEQ ID |
| | | | | NO: 4 | | NO: 7 | | | NO: 14 | NO: 21 |
| V11 | | GUSB | 1-418 | SEQ ID | 1110-1807 | SEQ ID | 698 | 419-1109 | SEQ ID | SEQ ID |
| | | | | NO: 5 | | NO: 8 | | | NO: 15 | NO: 22 |
| V12 | | GUSB | 1-418 | SEQ ID | 933-1807 | SEQ ID | 875 | 419-932 | SEQ ID | SEQ ID |
| | | | | NO: 5 | | NO: 9 | | | NO: 16 | NO: 23 |
| V13 | | GUSB | 1-418 | SEQ ID | 933-1807 | SEQ ID | 875 | 419-932 | SEQ ID | SEQ ID |
| | | | | NO: 5 | | NO: 9 | | | NO: 16 | NO: 23 |
| V14 | | GUSB | 1-418 | SEQ ID | 933-946 and | SEQ ID | 875 Δ25 | 419-932, | SEQ ID | SEQ ID |
| | | | | NO: 5 | 989-1807 | NO: 10 | | 947-988 | NO: 17 | NO: 24 |
| V15 | | GUSB | 1-418 | SEQ ID | 933-946, | SEQ ID | 875 Δ25 Δ30 | 419-932, | SEQ ID | SEQ ID |
| | | | | NO: 5 | 989-1204, | NO: 11 | | 947-988, | NO: 18 | NO: 25 |
| | | | | | 1272-1807 | | | 1205-1271 | | |
| V16 | | GUSB | 1-418 | SEQ ID | 933-946, | SEQ ID | 875 Δ25 Δ30 Δ33 | 419-932, | SEQ ID | SEQ ID |
| | | | | NO: 5 | 989-1204, | NO: 12 | | 947-988, | NO: 19 | NO: 26 |
| | | | | | 1272-1335, | | | 1205-1271, | | |
| | | | | | 1498-1807 | | | 1336-1497 | | |

AAV vectors, comprising AAV expression cassettes comprising the nucleic acid sequence encoding each of the cTuberins listed in Table 1, are generated. The AAV expression cassettes comprise a CB6 promoter with or without a CMV-IE upstream response element (URE) or a GUSB promoter operably linked to the nucleic acid sequence encoding each of the variants as indicated in Table 1. The AAV vector is packaged into AAV particles using the triple transfection method. In some cases, the AAV is a type 1 AAV (e.g., AAV1) or a type 9 AAV (e.g., AAV9). In some cases, the vectors include one or more ITR elements, such as one or more AAV2 ITR elements.

AAV vectors such as those described above can be prepared by any useful method. In some embodiments, AAV vectors are prepared using a cell culture (e.g., HEK293 suspension culture) transfected (e.g., triple transfected) to produce the product of interest. Subsequent processing may include one or more lysing, ion exchange chromatography, filtration (including ultrafiltration), affinity chromatography, and/or dilution steps. The sequence of vectors can be confirmed using various sequencing methods. The titer (e.g., gc/mL) can be determined using PCR amplification methods or other methods.

### Example 2: Expression of cTuberin in Cell Culture

Cells (e.g., COS-7 cells) are transfected with vector plasmids comprising nucleic acid sequences corresponding to the cTuberins described in Example 1. After 24 hours, expression of cTuberin is detected by immunoblotting (e.g., Western blotting) with an anti-Tuberin/TSC2 antibody.

To test the activity of cTuberin, cells (e.g., COS-7 cells) are transfected with various AAV constructs including, for example, GFP, pAAV-CBA-cTSC2, TSC2-FLAG, pAAV-CBA-cTSC2+TSC1-FLAG, TSC1-FLAG+TSC2-FLAG, and TSC1-FLAG vectors. Expression levels of phosphorylated S6 (pS6), S6, and GAPDH are detected by immunoblotting (e.g., Western blotting). While pS6 kinase levels are normally elevated in the absence of tuberin activity, cells transfected with a plasmid provided herein may show lower pS6 levels, which would indicate decreased pS6 kinase activity.

### Example 3: Expression of cTuberin In Vivo

The efficacy of the vectors of the present disclosure is further evaluated using mice lacking tuberin, such as Tcs2^{c/c} floxed mice described by Onda et al. (Onda et al., J. Clin. Invest. 104(6):687-695, 1999). In response to Cre recombinase, Tsc2^{c/c} alleles are converted to null alleles, and the lacZ allele expresses β-galactosidase. Such mice have been observed to have healthy and normal lifespans.

Intracerebroventricular (ICV) and/or retro-orbital (RO) injections are carried out using selected vectors described in Example 1. ICV injections are performed early in the lifecycle of the mice, such as an postnatal day (PND) 0, 1, 2, 3, 4, or 5. RO injections are performed later in the lifecycle of the mice, such as between PND 7-35, such as on PND 21. Injected titers can range from 1×10¹¹ genome copies per milliliter (gc/mL) to 1×10¹⁴ gc/mL. Survival is monitored. The brains of tuberin-lacking mice and treated mice are studied using hematoxylin and eosin (H&E) staining and/or immunohistochemical analysis for pS6.

In one study, the impact of cTuberin vectors such as those described in Example 1 on survival is evaluated. Tcs2c/c mice are injected ICV at PND0 or PND3 with a Cre carrying vector such as AAV1-CBA-Cre; injected RO at P21 with a cTuberin vector described in Example 1; or not injected. At PND3, the cerebral spinal fluid (CSF) barrier may be somewhat less penetrable than at P0, such that less loss of tuberin in the brain may be observed. Survival is monitored. Survival of mice injected with a cTuberin vector as expected to survive much longer than those injected with a Cre vector. Survival of mice injected with a cTuberin vector may be comparable to that of non-injected mice (e.g., >175 days).

In another study, the efficacy of cTuberin vectors such as those described in Example 1 is evaluated. All mice are injected ICV at PND0 or PND3 with a Cre carrying vector such as AAV1-CBA-Cre. A first group of mice are then injected RO with a cTuberin vector described in Example 1 at PND21, and a second group of mice are not injected again. The survival of mice injected with a cTuberin vector is expected to be longer than that for mice not injected with a cTuberin vector. Brains of tuberin-lacking mice and cTuberin treated mice are studied using H&E staining or IHC for pS6 after sacrifice (e.g., at PND27). For tuberin lacking mice, ependymal cell proliferation, enlargement of pyramidal cells in the hippocampus, a subependymal nodule, and multiple subependymal nodules and proliferation may be observed. For cTuberin treated mice, brain tissue may appear more similar to that for normal, non-injected (e.g., control) brains.

In another study, the efficacy of cTuberin vectors such as those described in Example 1 in combination with an anticonvulsant is evaluated. TSC2 mutations are associated with earlier onset and higher frequency of seizures. Accordingly, a combination of a cTuberin vector and an anticonvulsant may be effective in treating subjects with TSC2. All mice are injected with ICV at P0 or P3 with a Cre carrying vector such as AAV1-CBA-Cre. A first group of mice are then injected RO with a cTuberin vector described in Example 1 at PND3 or later, and a second group of mice are not injected again. A group of mice injected with a cTuberin vector are treated with an anticonvulsant such as vigabatrin (e.g., at 200 mg/kg) and another group of mice is not treated with the anticonvulsant. The survival of mice injected with a cTuberin vector and treated with the anticonvulsant is expected to be longer than that for mice not injected with a cTuberin vector and not treated with drug, mice not injected with a cTuberin vector and treated with drug, and mice injected with a cTuberin vector and not treated with an anticonvulsant.

In a further study, the efficacy of cTuberin vectors such as those described in Example 1 is tested in vivo on lymphangioleiomyomatosis (LAM) tumors injected subcutaneously in NOD-SCID II2R gamma (NSG) mice. TSC2 null, immortalized angiomyolipoma cells expressing Fluc are suspended in serum media, mixed with Matrigel, and implanted subcutaneously in the backs of NSG mice. After several (e.g., 4) weeks, mice are injected intraperitoneally with the Fluc substrate D-luciferin (LUCNA-1G) and signal detected with a spectrum and anesthesia system. Tumor volume is monitored via bioluminescence at, e.g., seeks 1, 4, 6, 9, and 14. Tumors are injected or not injected with cTuberin vectors at, e.g., weeks 4 and 9. By week 14, tumors injected with the cTuberin vector are expected to cease increasing in size, while non-injected tumors may continue to expand in volume.

Additional experimental details and related studies are described in, for example, International Patent Publication No. PCT/US2018/033247 filed May 17, 2018, which application is herein incorporated by reference in its entirety.

### NUMBERED EMBODIMENTS

Embodiment 1. A condensed tuberin (cTuberin), comprising (i) an N-terminal region capable of binding hamartin, and (ii) a C-terminal GTPase-activating protein (GAP) region, wherein the cTuberin lacks amino acid residues 419 to 932 of SEQ ID NO: 1.

Embodiment 2. The cTuberin of embodiment 1, wherein the cTuberin further lacks amino acid residues 947-988 of SEQ ID NO: 1.

Embodiment 3. The cTuberin of embodiment 1 or 2, wherein the cTuberin further lacks amino acid residues 1205-1271 of SEQ ID NO: 1.

Embodiment 4. The cTuberin of any one of embodiments 1-3, wherein the cTuberin further lacks amino acid residues 1336-1497 of SEQ ID NO: 1.

Embodiment 5. The cTuberin of embodiment 1, wherein the C-terminal region comprises an amino acid sequence with at least 90% identity to one of SEQ ID NOs: 10-12.

Embodiment 6. The cTuberin of embodiment 1, wherein the cTuberin further lacks amino acid residues 933 to 1109 of SEQ ID NO: 1.

Embodiment 7. The cTuberin of embodiment 6, wherein the C-terminal domain comprises an amino acid sequence with at least 90% identity to SEQ ID NO: 8.

Embodiment 8. The cTuberin of any one of embodiments 1-7, wherein the N-terminal domain comprises an amino acid sequence with at least 90% identity to SEQ ID NO: 5.

Embodiment 9. A condensed tuberin (cTuberin), comprising (i) an N-terminal region capable of binding hamartin, and (ii) a C-terminal GTPase-activating protein (GAP) region, wherein the C-terminal region comprises an amino acid sequence with at least 90% identity to SEQ ID NO: 7, and wherein the cTuberin lacks amino acid residues 451 to 932 of SEQ ID NO: 1.

Embodiment 10. The cTuberin of embodiment 9, wherein the cTuberin lacks amino acid residues 419 to 932 of SEQ ID NO: 1.

Embodiment 11. The cTuberin of embodiment 9 or 10, wherein the C-terminal region comprises an amino acid sequence with at least 90% identity to SEQ ID NO: 8.

Embodiment 12. The cTuberin of embodiment 9 or 10, wherein the C-terminal region comprises an amino acid sequence with at least 90% identity to SEQ ID NO: 9.

Embodiment 13. The cTuberin of embodiment 9, wherein the N-terminal region comprises an amino acid sequence with at least 90% identity to SEQ ID NO: 4.

Embodiment 14. The cTuberin of any one of embodiments 9-12, wherein the N-terminal region comprises an amino acid sequence with at least 90% identity to SEQ ID NO: 5.

Embodiment 15. The cTuberin of embodiment 9, wherein the cTuberin comprises an amino acid sequence with at least 90% identity to SEQ ID NO: 14.

Embodiment 16. The cTuberin of embodiment 9, wherein the cTuberin comprises an amino acid sequence with at least 90% identity to SEQ ID NO: 15.

Embodiment 17. The cTuberin of embodiment 9, wherein the cTuberin comprises an amino acid sequence with at least 90% identity to SEQ ID NO: 16.

Embodiment 18. A condensed tuberin (cTuberin), comprising (i) an N-terminal region capable of binding hamartin, and (ii) a C-terminal GTPase-activating protein (GAP) region, wherein the C-terminal region comprises an amino acid sequence with at least 90% identity to one of SEQ ID NOs: 10-12, and wherein the cTuberin lacks amino acid residues 451 to 932 of SEQ ID NO: 1.

Embodiment 19. The cTuberin of embodiment 18, wherein the cTuberin lacks amino acid residues 419-932 of SEQ ID NO: 1.

Embodiment 20. The cTuberin of embodiment 18 or 19, wherein the C-terminal region comprises an amino acid sequence with at least 90% identity to SEQ ID NO: 10.

Embodiment 21. The cTuberin of embodiment 18 or 19, wherein the C-terminal region comprises an amino acid sequence with at least 90% identity to SEQ ID NO: 11.

Embodiment 22. The cTuberin of embodiment 18 or 19, wherein the C-terminal region comprises an amino acid sequence with at least 90% identity to SEQ ID NO: 12.

Embodiment 23. The cTuberin of embodiment 18, wherein the cTuberin comprises an amino acid sequence with at least 90% identity to SEQ ID NO: 17.

Embodiment 24. The cTuberin of embodiment 18, wherein the cTuberin comprises an amino acid sequence with at least 90% identity to SEQ ID NO: 18.

Embodiment 25. The cTuberin of embodiment 18, wherein the cTuberin comprises an amino acid sequence with at least 90% identity to SEQ ID NO: 19.

Embodiment 26. The cTuberin of any one of embodiments 18-25, wherein the N-terminal region comprises an amino acid sequence with at least 90% identity to SEQ ID NO: 5.

Embodiment 27. The cTuberin of any one of embodiments 1-26, wherein the cTuberin comprises a spacer sequence between the N-terminal region and the C-terminal region.

Embodiment 28. The cTuberin of embodiment 27, wherein the spacer sequence comprises the sequence of SEQ ID NO: 2.

Embodiment 29. The cTuberin of embodiment 28, wherein the spacer sequence comprises the sequence of SEQ ID NO: 3.

Embodiment 30. A nucleic acid molecule encoding the cTuberin of any one of embodiments 1-29.

Embodiment 31. The nucleic acid molecule of embodiment 30, wherein the nucleic acid molecule is codon optimized for expression in a human target cell.

Embodiment 32. The nucleic acid molecule of embodiment 31, wherein the human target cell is a brain cell, heart cell, kidney cell, skin cell, or lung cell.

Embodiment 33. The nucleic acid molecule of any one of embodiments 30-32, wherein the nucleic acid molecule is operably linked to a regulatory control sequence.

Embodiment 34. The nucleic acid molecule of embodiment 33, wherein the regulatory control sequence comprises a human cytomegalovirus (CMV) promoter, a chicken β-actin (CBA) promoter, a Rous sarcoma virus (RSV) LTR promoter/enhancer, an SV40 promoter, a dihydrofolate reductase promoter, a phosphoglycerol kinase promoter, a CMV immediate/early gene enhancer/CBA promoter, a synapsin promoter, or a glial fibrillary acidic protein (GFAP) promoter.

Embodiment 35. The nucleic acid molecule of embodiment 33, wherein the regulatory control sequence comprises a human cytomegalovirus (CMV) immediate/early gene enhancer/chicken β-actin (CBA) promoter and a woodchuck hepatitis virus posttranscriptional regulatory element (WPRE).

Embodiment 36. The nucleic acid molecule of embodiment 33, wherein the regulatory control sequence comprises a beta-glucuronidase (GUSB) promoter.

Embodiment 37. The nucleic acid molecule of any one of embodiments 30-36, wherein the nucleic acid molecule has at least 90% sequence identity to any one of SEQ ID NOs. 21-26.

Embodiment 38. A nucleic acid molecule encoding a cTuberin comprising (i) an N-terminal region capable of binding hamartin, and (ii) a C-terminal GTPase-activating protein (GAP) region, wherein the cTuberin lacks amino acid residues 451 to 932 of SEQ ID NO: 1; and wherein the nucleic acid molecule is operably linked to a regulatory control sequence comprising a beta-glucuronidase (GUSB) promoter.

Embodiment 39. The nucleic acid molecule of embodiment 38, wherein the cTuberin lacks amino acid residues 451 to 1515 of SEQ ID NO: 1.

Embodiment 40. The nucleic acid molecule of embodiment 38 or 39, wherein the C-terminal region comprises an amino acid sequence with at least 90% identity to SEQ ID NO: 6.

Embodiment 41. The nucleic acid molecule of any one of embodiments 38-40, wherein the N-terminal region comprises an amino acid sequence with at least 90% identity to SEQ ID NO: 4.

Embodiment 42. A nucleic acid molecule, comprising an adeno-associated virus (AAV) expression cassette, the AAV expression cassette comprising from 5′ to 3′:
i) a 5′ AAV inverted terminal repeat (ITR);
ii) the nucleic acid molecule of any one of embodiments 25-35; and
iii) a 3′ AAV ITR.

Embodiment 43. The nucleic acid molecule of embodiment 42, wherein the 5′ ITR and/or the 3′ ITR are derived from AAV2.

Embodiment 44. The nucleic acid molecule of embodiment 42 or 43, wherein the 5′ AAV ITR sequence comprises a nucleic acid sequence with at least 90% identity to SEQ ID NO: 27.

Embodiment 45. The nucleic acid molecule of any one of embodiments 42-44, wherein the 3′ AAV ITR sequence comprises a nucleic acid sequence with at least 90% identity to SEQ ID NO: 28.

Embodiment 46. The nucleic acid molecule of any one of embodiments 42-45, wherein the AAV expression cassette further comprises a polyadenylation sequence.

Embodiment 47. The nucleic acid molecule of any one of embodiments 42-46, wherein the AAV expression cassette further comprises a Kozak sequence.

Embodiment 48. A plasmid, comprising the nucleic acid molecule of any one of embodiments 30-47.

Embodiment 49. A host cell, comprising the nucleic acid molecule of any one of embodiments 30-47, or the plasmid of embodiment 48.

Embodiment 50. A composition, comprising the nucleic acid molecule of any one of embodiments 30-47, the plasmid of embodiment 48, or the host cell of embodiment 49.

Embodiment 51. A method of producing a recombinant adeno-associated virus (rAAV), the method comprising: contacting a host cell with the nucleic acid molecule of any one of embodiments 30-47, or the plasmid of embodiment 48.

Embodiment 52. A recombinant adeno-associated virus (rAAV) produced by the method of embodiment 51.

Embodiment 53. A recombinant adeno-associated virus (rAAV), comprising: an AAV capsid protein; and the nucleic acid molecule of any one of embodiments 30-47.

Embodiment 54. The rAAV of embodiment 52 or 53, wherein the rAAV comprises an AAV1 capsid protein, an AAV2 capsid protein, an AAV3 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAVrh10 capsid protein, an AAV11 capsid protein, and/or an AAV12 capsid protein.

Embodiment 55. A method of expressing cTuberin in a target cell, comprising: contacting the target cell with the nucleic acid molecule of any one of embodiments 30-47, the plasmid of embodiment 48, the composition of embodiment 50, or the rAAV of any one of embodiments 52-54, thereby expressing cTuberin in the target cell.

Embodiment 56. The method of embodiment 55, wherein the contacting step is performed in vitro, ex vivo, or in vivo.

Embodiment 57. The method of embodiment 56, wherein the contacting step is performed in vivo in a subject in need thereof.

Embodiment 58. The method of embodiment 57, wherein the contacting step comprises administering a therapeutically effective amount of the nucleic acid molecule, the plasmid, the composition, or the rAAV to the subject.

Embodiment 59. A method of treating a subject having tuberous sclerosis complex (TSC), comprising: administering to the subject a therapeutically effective amount of the cTuberin of any one of embodiments 1-29, the nucleic acid molecule of any one of embodiments 30-47, one or more extracellular vesicles (EVs) comprising the nucleic acid molecule of any one of embodiments 30-47, the plasmid of embodiment 48, the composition of embodiment 50, or the rAAV of any one of embodiments 52-54, thereby treating TSC in the subject.

Embodiment 60. A method of treating a subject having renal cancer, comprising: administering to the subject a therapeutically effective amount of the cTuberin of any one of embodiments 1-29, the nucleic acid molecule of any one of embodiments 30-47, one or more extracellular vesicles (EVs) comprising the nucleic acid molecule of any one of embodiments 30-47, the plasmid of embodiment 48, the composition of embodiment 50, or the rAAV of any one of embodiments 52-54, thereby treating renal cancer in the subject.

Embodiment 61. The method of any one of embodiments 57-60, wherein the cTuberin, the nucleic acid molecule, the plasmid, the composition, or the rAAV is administered intravascularly, into the renal artery or vein, into the lungs, into the cisterna magna, intracerebrally, intrathecally, intravenously, intraventricularly, intracerebroventricularly, intraperitoneally, or dermally.

Embodiment 62. The method of any one of embodiments 57-61, wherein the subject has renal angiomyolipoma.

Embodiment 63. The method of embodiment 62, wherein the cTuberin, the nucleic acid molecule, the plasmid, the composition, or the rAAV is targeted to the renal angiomyolipoma.

Embodiment 64. The method of any one of embodiments 57-63, wherein the subject exhibits lymphangioleiomyomatosis (LAM).

Embodiment 65. The method of embodiment 64, wherein the cTuberin, the nucleic acid molecule, the plasmid, the composition, or the rAAV is targeted to the LAM.

Embodiment 66. The method of any one of embodiments 57-65, wherein the subject has a brain dysfunction.

Embodiment 67. The method of embodiment 66, wherein the cTuberin, the nucleic acid molecule, the plasmid, the composition, or the rAAV is provided to the subarachnoid space.

Embodiment 68. The method of any one of embodiments 57-67, wherein the cTuberin, the nucleic acid molecule, the plasmid, the composition, or the rAAV is administered to a brain cell, a heart cell, a kidney cell, a skin cell, or a lung cell.

Embodiment 69. The method of any one of embodiments 57-68, wherein the subject is administered rapamycin.

Embodiment 70. The method of any one of embodiments 57-69, wherein the subject is a human.

Embodiment 71. The method of any one of embodiments 57-70, wherein the subject is less than 18 years of age.

Embodiment 72. The method of embodiment 71, wherein the subject is an infant.

Embodiment 73. The method of any one of embodiments 57-72, wherein the subject has been diagnosed with tuberous sclerosis complex.

Embodiment 74. The method of any one of embodiments 57-73, wherein the subject has a mutation in the TSC2 gene.

Embodiment 75. The method of embodiment 74, wherein the subject has a mutation in exon 33, exon 37, and/or exon 38 of the TSC2 gene.

Embodiment 76. The method of any one of embodiments 57-75, wherein the subject has one or more of the following: cortical tubers, subependymal nodules, and subependymal giant cell astrocytomas.

1. A condensed tuberin (cTuberin), comprising (i) an N-terminal region capable of binding hamartin, and (ii) a C-terminal GTPase-activating protein (GAP) region, wherein the cTuberin lacks amino acid residues 419 to 932 of SEQ ID NO: 1.

2. The cTuberin of claim 1, wherein the cTuberin further lacks amino acid residues 947-988 of SEQ ID NO: 1.

3. The cTuberin of claim 1 or 2, wherein the cTuberin further lacks amino acid residues 1205-1271 of SEQ ID NO: 1.

4. The cTuberin of any one of claims 1-3, wherein the cTuberin further lacks amino acid residues 1336-1497 of SEQ ID NO: 1.

5. The cTuberin of claim 1, wherein the C-terminal region comprises an amino acid sequence with at least 90% identity to one of SEQ ID NOs: 10-12.

6. The cTuberin of claim 1, wherein the cTuberin further lacks amino acid residues 933 to 1109 of SEQ ID NO: 1.

7. The cTuberin of claim 6, wherein the C-terminal domain comprises an amino acid sequence with at least 90% identity to SEQ ID NO: 8.

8. The cTuberin of any one of claims 1-7, wherein the N-terminal domain comprises an amino acid sequence with at least 90% identity to SEQ ID NO: 5.

9. A condensed tuberin (cTuberin), comprising (i) an N-terminal region capable of binding hamartin, and (ii) a C-terminal GTPase-activating protein (GAP) region, wherein the C-terminal region comprises an amino acid sequence with at least 90% identity to SEQ ID NO: 7, and wherein the cTuberin lacks amino acid residues 451 to 932 of SEQ ID NO: 1.

10. The cTuberin of claim 9, wherein the cTuberin lacks amino acid residues 419 to 932 of SEQ ID NO: 1.

11. The cTuberin of claim 9 or 10, wherein the C-terminal region comprises an amino acid sequence with at least 90% identity to SEQ ID NO: 8.

12. The cTuberin of claim 9 or 10, wherein the C-terminal region comprises an amino acid sequence with at least 90% identity to SEQ ID NO: 9.

13. The cTuberin of claim 9, wherein the N-terminal region comprises an amino acid sequence with at least 90% identity to SEQ ID NO: 4.

14. The cTuberin of any one of claims 9-12, wherein the N-terminal region comprises an amino acid sequence with at least 90% identity to SEQ ID NO: 5.

15. The cTuberin of claim 9, wherein the cTuberin comprises an amino acid sequence with at least 90% identity to SEQ ID NO: 14.

16. The cTuberin of claim 9, wherein the cTuberin comprises an amino acid sequence with at least 90% identity to SEQ ID NO: 15.

17. The cTuberin of claim 9, wherein the cTuberin comprises an amino acid sequence with at least 90% identity to SEQ ID NO: 16.

18. A condensed tuberin (cTuberin), comprising (i) an N-terminal region capable of binding hamartin, and (ii) a C-terminal GTPase-activating protein (GAP) region, wherein the C-terminal region comprises an amino acid sequence with at least 90% identity to one of SEQ ID NOs: 10-12, and wherein the cTuberin lacks amino acid residues 451 to 932 of SEQ ID NO: 1.

19. The cTuberin of claim 18, wherein the cTuberin lacks amino acid residues 419-932 of SEQ ID NO: 1.

20. The cTuberin of claim 18 or 19, wherein the C-terminal region comprises an amino acid sequence with at least 90% identity to SEQ ID NO: 10.

21. The cTuberin of claim 18 or 19, wherein the C-terminal region comprises an amino acid sequence with at least 90% identity to SEQ ID NO: 11.

22. The cTuberin of claim 18 or 19, wherein the C-terminal region comprises an amino acid sequence with at least 90% identity to SEQ ID NO: 12.

23. The cTuberin of claim 18, wherein the cTuberin comprises an amino acid sequence with at least 90% identity to SEQ ID NO: 17.

24. The cTuberin of claim 18, wherein the cTuberin comprises an amino acid sequence with at least 90% identity to SEQ ID NO: 18.

25. The cTuberin of claim 18, wherein the cTuberin comprises an amino acid sequence with at least 90% identity to SEQ ID NO: 19.

26. The cTuberin of any one of claims 18-25, wherein the N-terminal region comprises an amino acid sequence with at least 90% identity to SEQ ID NO: 5.

27. The cTuberin of any one of claims 1-26, wherein the cTuberin comprises a spacer sequence between the N-terminal region and the C-terminal region.

28. The cTuberin of claim 27, wherein the spacer sequence comprises the sequence of SEQ ID NO: 2.

29. The cTuberin of claim 28, wherein the spacer sequence comprises the sequence of SEQ ID NO: 3.

30. A nucleic acid molecule encoding the cTuberin of any one of claims 1-29.

31. The nucleic acid molecule of claim 30, wherein the nucleic acid molecule is codon optimized for expression in a human target cell.

32. The nucleic acid molecule of claim 31, wherein the human target cell is a brain cell, heart cell, kidney cell, skin cell, or lung cell.

33. The nucleic acid molecule of any one of claims 30-32, wherein the nucleic acid molecule is operably linked to a regulatory control sequence.

34. The nucleic acid molecule of claim 33, wherein the regulatory control sequence comprises a human cytomegalovirus (CMV) promoter, a chicken β-actin (CBA) promoter, a Rous sarcoma virus (RSV) LTR promoter/enhancer, an SV40 promoter, a dihydrofolate reductase promoter, a phosphoglycerol kinase promoter, a CMV immediate/early gene enhancer/CBA promoter, a synapsin promoter, or a glial fibrillary acidic protein (GFAP) promoter.

35. The nucleic acid molecule of claim 33, wherein the regulatory control sequence comprises a human cytomegalovirus (CMV) immediate/early gene enhancer/chicken β-actin (CBA) promoter and a woodchuck hepatitis virus posttranscriptional regulatory element (WPRE).

36. The nucleic acid molecule of claim 33, wherein the regulatory control sequence comprises a beta-glucuronidase (GUSB) promoter.

37. The nucleic acid molecule of any one of claims 30-36, wherein the nucleic acid molecule has at least 90% sequence identity to any one of SEQ ID NOs. 21-26.

38. A nucleic acid molecule encoding a cTuberin comprising (i) an N-terminal region capable of binding hamartin, and (ii) a C-terminal GTPase-activating protein (GAP) region, wherein the cTuberin lacks amino acid residues 451 to 932 of SEQ ID NO: 1; and wherein the nucleic acid molecule is operably linked to a regulatory control sequence comprising a beta-glucuronidase (GUSB) promoter.

39. The nucleic acid molecule of claim 38, wherein the cTuberin lacks amino acid residues 451 to 1515 of SEQ ID NO: 1.

40. The nucleic acid molecule of claim 38 or 39, wherein the C-terminal region comprises an amino acid sequence with at least 90% identity to SEQ ID NO: 6.

41. The nucleic acid molecule of any one of claims 38-40, wherein the N-terminal region comprises an amino acid sequence with at least 90% identity to SEQ ID NO: 4.

42. A nucleic acid molecule, comprising an adeno-associated virus (AAV) expression cassette, the AAV expression cassette comprising from 5′ to 3′:
i) a 5′ AAV inverted terminal repeat (ITR);
ii) the nucleic acid molecule of any one of claims 25-35; and
iii) a 3′ AAV ITR.

43. The nucleic acid molecule of claim 42, wherein the 5′ ITR and/or the 3′ ITR are derived from AAV2.

44. The nucleic acid molecule of claim 42 or 43, wherein the 5′ AAV ITR sequence comprises a nucleic acid sequence with at least 90% identity to SEQ ID NO: 27.

45. The nucleic acid molecule of any one of claims 42-44, wherein the 3′ AAV ITR sequence comprises a nucleic acid sequence with at least 90% identity to SEQ ID NO: 28.

46. The nucleic acid molecule of any one of claims 42-45, wherein the AAV expression cassette further comprises a polyadenylation sequence.

47. The nucleic acid molecule of any one of claims 42-46, wherein the AAV expression cassette further comprises a Kozak sequence.

48. A plasmid, comprising the nucleic acid molecule of any one of claims 30-47.

49. A host cell, comprising the nucleic acid molecule of any one of claims 30-47, or the plasmid of claim 48.

50. A composition, comprising the nucleic acid molecule of any one of claims 30-47, the plasmid of claim 48, or the host cell of claim 49.

51. A method of producing a recombinant adeno-associated virus (rAAV), the method comprising: contacting a host cell with the nucleic acid molecule of any one of claims 30-47, or the plasmid of claim 48.

52. A recombinant adeno-associated virus (rAAV) produced by the method of claim 51.

53. A recombinant adeno-associated virus (rAAV), comprising: an AAV capsid protein; and the nucleic acid molecule of any one of claims 30-47.

54. The rAAV of claim 52 or 53, wherein the rAAV comprises an AAV1 capsid protein, an AAV2 capsid protein, an AAV3 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAVrh10 capsid protein, an AAV11 capsid protein, and/or an AAV12 capsid protein.

55. A method of expressing cTuberin in a target cell, comprising: contacting the target cell with the nucleic acid molecule of any one of claims 30-47, the plasmid of claim 48, the composition of claim 50, or the rAAV of any one of claims 52-54, thereby expressing cTuberin in the target cell.

56. The method of claim 55, wherein the contacting step is performed in vitro, ex vivo, or in vivo.

57. The method of claim 56, wherein the contacting step is performed in vivo in a subject in need thereof.

58. The method of claim 57, wherein the contacting step comprises administering a therapeutically effective amount of the nucleic acid molecule, the plasmid, the composition, or the rAAV to the subject.

59. A method of treating a subject having tuberous sclerosis complex (TSC), comprising: administering to the subject a therapeutically effective amount of the cTuberin of any one of claims 1-29, the nucleic acid molecule of any one of claims 30-47, one or more extracellular vesicles (EVs) comprising the nucleic acid molecule of any one of claims 30-47, the plasmid of claim 48, the composition of claim 50, or the rAAV of any one of claims 52-54, thereby treating TSC in the subject.

60. A method of treating a subject having renal cancer, comprising: administering to the subject a therapeutically effective amount of the cTuberin of any one of claims 1-29, the nucleic acid molecule of any one of claims 30-47, one or more extracellular vesicles (EVs) comprising the nucleic acid molecule of any one of claims 30-47, the plasmid of claim 48, the composition of claim 50, or the rAAV of any one of claims 52-54, thereby treating renal cancer in the subject.

61. The method of any one of claims 57-60, wherein the cTuberin, the nucleic acid molecule, the plasmid, the composition, or the rAAV is administered intravascularly, into the renal artery or vein, into the lungs, into the cisterna magna, intracerebrally, intrathecally, intravenously, intraventricularly, intracerebroventricularly, intraperitoneally, or dermally.

62. The method of any one of claims 57-61, wherein the subject has renal angiomyolipoma.

63. The method of claim 62, wherein the cTuberin, the nucleic acid molecule, the plasmid, the composition, or the rAAV is targeted to the renal angiomyolipoma.

64. The method of any one of claims 57-63, wherein the subject exhibits lymphangioleiomyomatosis (LAM).

65. The method of claim 64, wherein the cTuberin, the nucleic acid molecule, the plasmid, the composition, or the rAAV is targeted to the LAM.

66. The method of any one of claims 57-65, wherein the subject has a brain dysfunction.

67. The method of claim 66, wherein the cTuberin, the nucleic acid molecule, the plasmid, the composition, or the rAAV is provided to the subarachnoid space.

68. The method of any one of claims 57-67, wherein the cTuberin, the nucleic acid molecule, the plasmid, the composition, or the rAAV is administered to a brain cell, a heart cell, a kidney cell, a skin cell, or a lung cell.

69. The method of any one of claims 57-68, wherein the subject is administered rapamycin.

70. The method of any one of claims 57-69, wherein the subject is a human.

71. The method of any one of claims 57-70, wherein the subject is less than 18 years of age.

72. The method of claim 71, wherein the subject is an infant.

73. The method of any one of claims 57-72, wherein the subject has been diagnosed with tuberous sclerosis complex.

74. The method of any one of claims 57-73, wherein the subject has a mutation in the TSC2 gene.

75. The method of claim 74, wherein the subject has a mutation in exon 33, exon 37, and/or exon 38 of the TSC2 gene.

76. The method of any one of claims 57-75, wherein the subject has one or more of the following: cortical tubers, subependymal nodules, and subependymal giant cell astrocytomas.
